(19) United States
(12) Patent Application Publication
(10) Pub. No.: US 2026/0265387 A1
(43) Pub. Date: Sep. 10, 2026

(54) ANTI-TSHR ANTIBODIES AND USES THEREOF

(71) Applicant: ETHYREAL BIO, INC., Cambridge, MA (US)

(72) Inventors: Lauren Pepper, Belmont, MA (US); Charles Gordon Grummitt, Bagunte (PT); Alexey Alexandrovich Lugovskoy, Belmont, MA (US); Melissa Geddie, Arlington, MA (US)

(21) Appl. No.: 19/558,850

(22) Filed: Mar. 6, 2026

Related U.S. Application Data
(60) Provisional application No. 63/768,482, filed on Mar. 7, 2025.
(60) Provisional application No. 63/964,648, filed on Jan. 21, 2026.

(30) Foreign Application Priority Data
Mar. 27, 2025 (EP) 25166758.0

(51) Int. Cl. C07K 16/28; A61K 39/00; A61K 47/68; A61K 51/10; A61P 5/14

(52) U.S. Cl. CPC C07K 16/2869; A61K 47/6801; A61K 47/6849; A61K 51/1036; A61K 51/1093; A61P 5/14; A61K 2039/505; C07K 2317/34; C07K 2317/565; C07K 2317/76; C07K 2317/92; C07K 2317/94

(57) ABSTRACT

Provided herein are antigen binding proteins that bind to thyroid stimulating hormone receptor (TSHR) and methods of using the same.

### RELATED APPLICATIONS

This application claims the benefit of U.S. Provisional Application Ser. No. 63/768,482, filed Mar. 7, 2025, European Priority Application 25166758.0, filed Mar. 27, 2025, and U.S. Provisional Application Ser. No. 63/964,648, filed Jan. 21, 2026, the entire disclosure of each are incorporated herein by reference.

### SEQUENCE LISTING

The instant application contains a Sequence Listing which has been submitted electronically in XML format and is hereby incorporated by reference in its entirety. Said XML file, created on Mar. 6, 2026, is named 773077_TB9-005_ST26.xml and is 71,268 bytes in size.

### BACKGROUND

Thyroid-stimulating hormone receptor (TSHR) is a G-protein coupled receptor (GPCR) that is mainly expressed on thyroid epithelial cells as well as periorbital fibroblasts and adipose tissue and has a key role in the regulation of thyroid function. Activation of TSHR by thyroid-stimulating hormone (TSH) results in the growth and proliferation of thyrocytes and thyroid hormone production, in particular the production of thyroxine (T₄) and triidothyronine (T₃). These hormones have several critical functions in the human body, as they support human metabolic and cardiovascular systems, and regulate the metabolism of fats, proteins, and carbohydrates. There are presently a number of known but unresolved problems relating to the thyroid including general thyroid disease and thyroid-related disorders (e.g., Graves' disease) as well as other diseases where TSHR plays a pathophysiological role (e.g., thyroid eye disease and thyroid cancer). Therefore, there is a need for therapeutics that prevent or treat these diseases and disorders associated with inappropriate TSHR activation.

### SUMMARY

The present disclosure provides antigen binding proteins which specifically bind to thyroid-stimulating hormone receptor (TSHR). Certain anti-TSHR antigen binding proteins described herein possess reduced off-target binding to CD80 relative to a parental anti-TSHR antibody while maintaining a similar binding affinity and activity. Alternative pH sensitive anti-TSHR antigen binding proteins described herein possess altered binding affinity between neutral (e.g., 7.4) and acidic (e.g., 5.8) pH. Said pH sensitive anti-TSHR antigen binding proteins exhibit substantially reduced affinity and faster off-rate at acidic pH.

In an aspect, provided herein is an antigen binding protein or an antigen binding fragment thereof which specifically binds thyroid-stimulating hormone receptor (TSHR), wherein the antigen binding protein or an antigen binding fragment thereof comprises a heavy chain variable (VH) domain and a light chain variable (VL) domain, wherein: a. the VH domain comprises a HCDR1 sequence comprising the amino acid sequence of SEQ ID NO: 1, a HCDR2 sequence comprising the amino acid sequence of SEQ ID NO: 2, and a HCDR3 sequence comprising the amino acid sequence of SEQ ID NO: 3; and the VL domain comprises a LCDR1 sequence comprising the amino acid sequence of SEQ ID NO: 4, a LCDR2 sequence comprising the amino acid sequence of SEQ ID NO: 5, and a LCDR3 sequence comprising the amino acid sequence of SEQ ID NO: 6; b. the VH domain comprises a HCDR1 sequence comprising the amino acid sequence of SEQ ID NO: 11, a HCDR2 sequence comprising the amino acid sequence of SEQ ID NO: 12, and a HCDR3 sequence comprising the amino acid sequence of SEQ ID NO: 13; and the VL domain comprises a LCDR1 sequence comprising the amino acid sequence of SEQ ID NO: 14, a LCDR2 sequence comprising the amino acid sequence of SEQ ID NO: 15, and a LCDR3 sequence comprising the amino acid sequence of SEQ ID NO: 16; c. the VH domain comprises a HCDR1 sequence comprising the amino acid sequence of SEQ ID NO: 21, a HCDR2 sequence comprising the amino acid sequence of SEQ ID NO: 22, and a HCDR3 sequence comprising the amino acid sequence of SEQ ID NO: 23; and the VL domain comprises a LCDR1 sequence comprising the amino acid sequence of SEQ ID NO: 24, a LCDR2 sequence comprising the amino acid sequence of SEQ ID NO: 25, and a LCDR3 sequence comprising the amino acid sequence of SEQ ID NO: 26; d. the VH domain comprises a HCDR1 sequence comprising the amino acid sequence of SEQ ID NO: 31, a HCDR2 sequence comprising the amino acid sequence of SEQ ID NO: 32, and a HCDR3 sequence comprising the amino acid sequence of SEQ ID NO: 33; and the VL domain comprises a LCDR1 sequence comprising the amino acid sequence of SEQ ID NO: 34, a LCDR2 sequence comprising the amino acid sequence of SEQ ID NO: 35, and a LCDR3 sequence comprising the amino acid sequence of SEQ ID NO: 36; e. the VH domain comprises a HCDR1 sequence comprising the amino acid sequence of SEQ ID NO: 41, a HCDR2 sequence comprising the amino acid sequence of SEQ ID NO: 42, and a HCDR3 sequence comprising the amino acid sequence of SEQ ID NO: 43; and the VL domain comprises a LCDR1 sequence comprising the amino acid sequence of SEQ ID NO: 44, a LCDR2 sequence comprising the amino acid sequence of SEQ ID NO: 45, and a LCDR3 sequence comprising the amino acid sequence of SEQ ID NO: 46; or f. the VH domain comprises a HCDR1 sequence comprising the amino acid sequence of SEQ ID NO: 51, a HCDR2 sequence comprising the amino acid sequence of SEQ ID NO: 52, and a HCDR3 sequence comprising the amino acid sequence of SEQ ID NO: 53; and the VL domain comprises a LCDR1 sequence comprising the amino acid sequence of SEQ ID NO: 54, a LCDR2 sequence comprising the amino acid sequence of SEQ ID NO: 55, and a LCDR3 sequence comprising the amino acid sequence of SEQ ID NO: 56.

In certain embodiments, a. the VH comprises an amino acid sequence at least 90% identical to SEQ ID NO: 7 and the VL comprises an amino acid sequence at least 90% identical to SEQ ID NO: 8; b. the VH comprises an amino acid sequence at least 90% identical to SEQ ID NO: 17 and the VL comprises an amino acid sequence at least 90% identical to SEQ ID NO: 18; c. the VH comprises an amino acid sequence at least 90% identical to SEQ ID NO: 27 and the VL comprises an amino acid sequence at least 90% identical to SEQ ID NO: 28; d. the VH comprises an amino acid sequence at least 90% identical to SEQ ID NO: 37 and the VL comprises an amino acid sequence at least 90% identical to SEQ ID NO: 38; e. the VH comprises an amino acid sequence at least 90% identical to SEQ ID NO: 47 and the VL comprises an amino acid sequence at least 90% identical to SEQ ID NO: 48; or f. the VH comprises an amino acid sequence at least 90% identical to SEQ ID NO: 57 and the VL comprises an amino acid sequence at least 90% identical to SEQ ID NO: 58.

In certain embodiments, a. the VH comprises an amino acid sequence of SEQ ID NO: 7 and the VL comprises an amino acid sequence of SEQ ID NO: 8; b. the VH comprises an amino acid sequence of SEQ ID NO: 17 and the VL comprises an amino acid sequence of SEQ ID NO: 18; c. the VH comprises an amino acid sequence of SEQ ID NO: 27 and the VL comprises an amino acid sequence of SEQ ID NO: 28; d. the VH comprises an amino acid sequence of SEQ ID NO: 37 and the VL comprises an amino acid sequence of SEQ ID NO: 38; e. the VH comprises an amino acid sequence of SEQ ID NO: 47 and the VL comprises an amino acid sequence of SEQ ID NO: 48; or f. the VH comprises an amino acid sequence of SEQ ID NO: 57 and the VL comprises an amino acid sequence of SEQ ID NO: 58.

In certain embodiments, the antigen binding protein or the antigen binding fragment thereof further comprises an immunoglobulin Fc domain or variant thereof.

In certain embodiments, the Fc domain or variant thereof comprises a first Fc heavy chain and a second Fc heavy chain.

In certain embodiments, one or both of the first Fc heavy chain and second Fc heavy chain comprises one or more mutations to increase serum half-life.

In certain embodiments, one or both of the first Fc heavy chain and second Fc heavy chain comprises one or more substitutions at amino acid positions 252, 254, and/or 256, according to EU numbering. In certain embodiments, the substitution at amino acid position 252 is a tyrosine (Y), the substitution at amino acid position 254 is a threonine (T), and the substitution at amino acid position 256 is a glutamic acid (E).

In certain embodiments, the Fc heavy chain comprises an amino acid sequence of SEQ ID NO: 69.

In certain embodiments, the antigen binding protein or the antigen binding fragment thereof comprises: a. a heavy chain polypeptide comprising an amino acid sequence at least 90% identical to SEQ ID NO: 9, and a light chain polypeptide comprising an amino acid sequence at least 90% identical to SEQ ID NO: 10; b. a heavy chain polypeptide comprising an amino acid sequence at least 90% identical to SEQ ID NO: 19, and a light chain polypeptide comprising an amino acid sequence at least 90% identical to SEQ ID NO: 20; c. a heavy chain polypeptide comprising an amino acid sequence at least 90% identical to SEQ ID NO: 29, and a light chain polypeptide comprising an amino acid sequence at least 90% identical to SEQ ID NO: 30; d. a heavy chain polypeptide comprising an amino acid sequence at least 90% identical to SEQ ID NO: 39, and a light chain polypeptide comprising an amino acid sequence at least 90% identical to SEQ ID NO: 40; e. a heavy chain polypeptide comprising an amino acid sequence at least 90% identical to SEQ ID NO: 49, and a light chain polypeptide comprising an amino acid sequence at least 90% identical to SEQ ID NO: 50; or f. a heavy chain polypeptide comprising an amino acid sequence at least 90% identical to SEQ ID NO: 59, and a light chain polypeptide comprising an amino acid sequence at least 90% identical to SEQ ID NO: 60.

In certain embodiments, the antigen binding protein or the antigen binding fragment thereof comprises: a. a heavy chain polypeptide comprising SEQ ID NO: 9, and a light chain polypeptide comprising SEQ ID NO: 10; b. a heavy chain polypeptide comprising SEQ ID NO: 19, and a light chain polypeptide comprising SEQ ID NO: 20; c. a heavy chain polypeptide comprising SEQ ID NO: 29, and a light chain polypeptide comprising SEQ ID NO: 30; d. a heavy chain polypeptide comprising SEQ ID NO: 39, and a light chain polypeptide comprising SEQ ID NO: 40; e. a heavy chain polypeptide comprising SEQ ID NO: 49, and a light chain polypeptide comprising SEQ ID NO: 50; or f. a heavy chain polypeptide comprising SEQ ID NO: 59, and a light chain polypeptide comprising SEQ ID NO: 60.

In certain embodiments, one or both of the first Fc heavy chain and second Fc heavy chain comprises one or more substitutions at amino acid positions 428 and/or 434, according to EU numbering.

In certain embodiments, the substitution at amino acid position 428 is a leucine (L), and wherein the substitution at amino acid position 434 is a serine (S).

In certain embodiments, the antigen binding protein or antigen binding fragment thereof has reduced binding to CD80 compared to an antigen binding protein comprising a VH domain of SEQ ID NO: 61 and a VL domain of SEQ ID NO: 62.

In certain embodiments, the antigen binding protein or antigen binding fragment thereof has at least about 2-fold reduced binding to CD80 compared to an antigen binding protein comprising a VH domain of SEQ ID NO: 61 and a VL domain of SEQ ID NO: 62.

In certain embodiments, binding to CD80 is determined by detecting binding of the antigen binding protein or the antigen binding fragment thereof to a cell that overexpresses CD80.

In certain embodiments, the antigen binding protein or the antigen binding fragment thereof has a higher melting temperature compared to an antigen binding protein comprising a VH domain of SEQ ID NO: 61 and a VL domain of SEQ ID NO: 62.

In certain embodiments, the antigen binding protein or the antigen binding fragment thereof has a melting temperature that is at least about 2° C. higher compared to an antigen binding protein comprising a VH domain of SEQ ID NO: 61 and a VL domain of SEQ ID NO: 62.

In certain embodiments, the melting temperature is determined by differential scanning fluorimetry (DSF).

In certain embodiments, the antigen binding protein or the antigen binding fragment thereof blocks TSHR autoantibodies from binding to TSHR.

In certain embodiments, the antigen binding protein or the antigen binding fragment thereof decreases an autoimmune antibody response compared to an antigen binding protein comprising a VH domain of SEQ ID NO: 61 and a VL domain of SEQ ID NO: 62.

In certain embodiments, the antigen binding protein or the antigen binding fragment thereof has a reduced anti-drug antibody response compared to an antigen binding protein comprising a VH domain of SEQ ID NO: 61 and a VL domain of SEQ ID NO: 62.

In certain embodiments, the antigen binding protein or the antigen binding fragment thereof has reduced immunogenicity compared to an antigen binding protein comprising a VH domain of SEQ ID NO: 61 and a VL domain of SEQ ID NO: 62.

In certain embodiments, the immunogenicity is determined by measuring the number of HLA class II restricted epitopes in the antigen binding protein or the antigen binding fragment thereof. In certain embodiments, the HLA class II restricted epitopes are any one or more of HLA-DRB1, HLA-DRB3, HLA-DRB4, HLA-DRB5, HLA-DQ, and HLA-DP.

In one aspect, the disclosure provides a pharmaceutical composition comprising the antigen binding protein or the antigen binding fragment thereof described herein and a pharmaceutically acceptable carrier.

In one aspect, the disclosure provides an isolated nucleic acid molecule encoding the antigen binding protein or the antigen binding fragment thereof described herein.

In one aspect, the disclosure provides an isolated nucleic acid molecule encoding the heavy chain variable (VH) domain described herein.

In one aspect, the disclosure provides an isolated nucleic acid molecule encoding the light chain variable (VL) domain described herein.

In certain embodiments, a first isolated nucleic acid molecule encodes a heavy chain polypeptide comprising SEQ ID NO: 29, and a second isolated nucleic acid molecule encodes a light chain polypeptide comprising SEQ ID NO: 30.

In certain embodiments, a first isolated nucleic acid molecule encodes a heavy chain polypeptide comprising SEQ ID NO: 9, and a second isolated nucleic acid molecule encodes a light chain polypeptide comprising SEQ ID NO: 10.

In certain embodiments, a first isolated nucleic acid molecule encodes a heavy chain polypeptide comprising SEQ ID NO: 19, and a second isolated nucleic acid molecule encodes a light chain polypeptide comprising SEQ ID NO: 20.

In certain embodiments, a first isolated nucleic acid molecule encodes a heavy chain polypeptide comprising SEQ ID NO: 39, and a second isolated nucleic acid molecule encodes a light chain polypeptide comprising SEQ ID NO: 40.

In certain embodiments, a first isolated nucleic acid molecule encodes a heavy chain polypeptide comprising SEQ ID NO: 49, and a second isolated nucleic acid molecule encodes a light chain polypeptide comprising SEQ ID NO: 50.

In certain embodiments, a first isolated nucleic acid molecule encodes a heavy chain polypeptide comprising SEQ ID NO: 69, and a second isolated nucleic acid molecule encodes a light chain polypeptide comprising SEQ ID NO: 70.

In one aspect, the disclosure provides an expression vector comprising the nucleic acid molecule described herein.

In one aspect, the disclosure provides a host cell comprising the expression vector described herein.

In one aspect, the disclosure provides a method of treating or preventing a thyroid stimulating hormone receptor (TSHR)-related disease in a subject, comprising administering to a subject in need thereof the antigen binding protein or antigen binding fragment thereof described herein, or the pharmaceutical composition described herein, or the CAR described herein, or the cell described herein, or the ADC described herein, thereby treating or preventing the TSHR-related disease in the subject.

In certain embodiments, the TSHR-related disease is an autoimmune disease.

In certain embodiments, the TSHR-related disease is Graves' disease.

In certain embodiments, the TSHR-related disease is thyrotoxicosis.

In certain embodiments, the TSHR-related disease is cancer.

In one aspect, the disclosure provides a method of treating an autoimmune disease associated with autoantibodies to thyroid stimulating hormone receptor (TSHR) in a subject, comprising administering to a subject in need thereof the antigen binding protein or antigen binding fragment thereof described herein or the pharmaceutical composition described herein, or the CAR described herein, or the cell described herein, or the ADC described herein, thereby treating or preventing the autoimmune disease associated with autoantibodies to TSHR in the subject.

In one aspect, the disclosure provides a chimeric antigen receptor (CAR) comprising a TSHR-binding domain comprising the antigen binding protein or antigen binding fragment thereof described herein.

In one aspect, the disclosure provides a cell comprising the CAR described herein.

In one aspect, the disclosure provides an antibody drug conjugate (ADC) comprising the antigen binding protein or an antigen binding fragment thereof described herein conjugated to a drug. In certain embodiments, the drug is a radioisotope or to a therapeutic small molecule.

### BRIEF DESCRIPTION OF THE DRAWINGS

FIG. 1A-FIG. 1B depicts an alignment of the parental VH and VL (ETY-1) against the germlined (ETY-2) and framework swapped (ETY-3) variants. The VH alignment is depicted in FIG. 1A and the VL alignment is depicted in FIG. 1B.

FIG. 2 depicts relative CD80 binding of select antibody variants compared to control antibodies in CHO cells overexpressing CD80 compared to WT CHO cells. Results are plotted based on mean fluorescent intensity (MFI).

FIG. 3 depicts relative CD80 binding of select antibody variants compared to control antibodies in HEK cells overexpressing CD80 compared to WT HEK cells. Results are plotted based on MFI.

FIG. 4 depicts relative CD80 binding of select antibody variants compared to control antibodies in HEK cells stably overexpressing CD80 compared to WT HEK cells. Results are plotted based on MFI.

FIG. 5 depicts relative CD80 binding of select antibody variants compared to control antibodies in a DELFIA (dissociation-enhanced lanthanide fluorescent immunoassay) assay. Results are plotted based on Time Resolved Fluorescence (TRF) signal.

FIG. 6 depicts cAMP concentration (nM) from cells incubated with select variant antibodies or the parental antibody over a range of concentrations.

FIG. 7A-FIG. 7F depicts membrane proteome array binding of ETY-77, ETY-86, ETY-94, ETY-98, ETY-155, and the parental antibody. Binding interactions confirmed in downstream validation studies are displayed by name of protein and any proteins that did not pass validation were removed.

FIG. 8 depicts cAMP concentration (pmol/mL) from cells incubated with TED patient sera having varied TSHR autoantibody concentrations. Sera samples were incubated with cells with or without ETY-94.

FIG. 9 depicts hyaluronic acid (HA) and IL-6 levels following incubation of primary human orbital fibroblasts from thyroid eye disease (TED) orbital tissue with a stimulatory anti-TSHR monoclonal antibody M22 (10-30 nM), alone or in combination with pharmacologic inhibitors (Teprotumumab, K1-70, or ETY-94). * P≤0.05; ** P≤0.01; *** P≤0.001; **** P≤0.0001.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

Although any methods and materials similar or equivalent to those described herein can be used in the practice of the present disclosure, exemplary methods and materials are now described. All publications mentioned herein are incorporated herein by reference to describe in their entirety.

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric. A polypeptide can have the amino acid sequence of naturally occurring polypeptide from any mammal. Such native sequence polypeptide can be isolated from nature or can be produced by recombinant or synthetic means. In some embodiments, the polypeptide is a “variant”. “Variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide. A “derivative” of a polypeptide is a polypeptide (e.g., an antibody) that has been chemically modified, e.g., via conjugation to another chemical moiety (such as, for example, polyethylene glycol or albumin, e.g., human serum albumin), phosphorylation, and glycosylation

As used herein, the terms “antibody” and “antibodies” include full-length antibodies, antigen binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, common light chain antibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. As used herein, the terms “VH” and “VL” refer to antibody heavy and light chain variable domain, respectively, as described in Kabat et al., (1991) Sequences of Proteins of Immunological Interest (NIH Publication No. 91-3242, Bethesda), which is herein incorporated by reference in its entirety.

As used herein, the term “antigen binding protein” or “binding domain” or “binding specificity” refers to a molecule that specifically binds to an antigen as such binding is understood by one skilled in the art. For example, an antigen binding protein that specifically binds to an antigen may bind to other molecules, generally with lower affinity as determined by, e.g., immunoassays (e.g., ELISA), BIAcore®, KinExA 3000 instrument (Sapidyne Instruments, Boise, ID), surface plasmon resonance (SPR) analysis, biolayer interferometry (BLI), or other assays known in the art. In certain embodiments, an antigen-binding moiety that specifically binds to an antigen binds to the antigen with a Ka that is at least 2 logs (e.g., factors of 10), 2.5 logs, 3 logs, 4 logs or greater than the Ka when the molecule binds non-specifically to another antigen.

As used herein, the term “VH/VL pair” refers to a combination of a VH and a VL that together form the binding site for an antigen.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4.

As used herein, the term “full-length antibody heavy chain” refers to an antibody heavy chain comprising, from N to C terminal, a VH, a CH1 region, a hinge region, a CH2 domain and a CH3 domain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain. As used herein, the term “complementarity determining region” or “CDR” refers to sequences of amino acids within antibody variable regions, which confer antigen specificity and binding affinity. In general, there are three CDRs in each heavy chain variable region (HCDR1, HCDR2, HCDR3) and three CDRs in each light chain variable region (LCDR1, LCDR2, LCDR3). Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th ed. (1991)). Thus, the VHs may be comprised within the corresponding CDRs and references herein to the “hypervariable loops” of VH and VL domains should be interpreted as also encompassing the corresponding CDRs, and vice versa, unless otherwise indicated. “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M. P. et al., “IMGT unique numbering for immunoglobulin and T cell receptor variable domains and Ig superfamily V-like domains,” Dev. Comp. Immunol., 2003 January; 27(1):55-77 (“IMGT” numbering scheme), and Honegger A. and Pluckthun A., “Yet another numbering scheme for immunoglobulin variable domains: an automatic modeling and analysis tool,” J. Mol. Biol., 2001 Jun. 8; 309(3):657-70, (Aho numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

As used herein, the term “single chain variable fragment” (scFv) refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “human antibody,” as used herein, is intended to include antibodies having variable and Fc domains derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “multispecific antigen-binding molecules,” as used herein refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. In certain embodiment, the multispecific antigen binding molecules of the disclosure comprises at least a first binding specificity for a subunit of a receptor and at least a second binding specificity for another receptor subunit. A multispecific antigen-binding molecule can be a single multifunctional polypeptide, or it can be a multimeric complex of two or more polypeptides that are covalently or non-covalently associated with one another. The term “multispecific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In certain exemplary embodiments, an antibody of the present disclosure is functionally linked to another antibody or antigen-binding fragment thereof to produce a bispecific antibody with a second binding specificity.

In exemplary embodiments, the heteromeric antibodies of the present disclosure are bispecific antibodies. Bispecific antibodies can be monoclonal, e.g., human or humanized, antibodies that have binding specificities for at least two different antigens. In certain embodiments, the bispecific antibodies of the disclosure comprises at least a first binding domain for a receptor subunit and at least a second binding domain for another receptor subunit.

Methods for making bispecific antibodies are well-known. Traditionally, the recombinant production of bispecific antibodies was based on the co-expression of two immunoglobulin heavy chain/light chain pairs, where the two heavy chains have different specificities (Milstein et al., Nature 305:537 (1983)). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. More modern techniques for generating bispecific antibodies employ heterodimerization domains that favor desired pairing of heavy chain from the antibody with a first specificity to the heavy chain of an antibody with a second specificity.

Antibody variable domains with the desired binding specificities can be fused to immunoglobulin constant domain sequences. The fusion typically is with an immunoglobulin heavy chain constant domain, comprising at least part of the hinge, CH2, and CH3 regions. It may have the first heavy chain Fc domain (CH1) containing the site necessary for light chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. Enzymol. 121:210 (1986).

As used herein, the term “Fc” refers to a polypeptide comprising a CH2 domain and a CH3 domain, wherein the C-terminus of the CH2 domain is linked (directly or indirectly) to the N-terminus of the CH3 domain. The term “Fc polypeptide” includes an antibody heavy chain linked to an antibody light chain by disulfide bonds (e.g., to form a half-antibody).

In certain embodiments, an Fc chain begins in the hinge region just upstream of the papain cleavage site and ends at the C-terminus of the antibody. Accordingly, a complete Fc chain comprises at least a hinge domain, a CH2 domain, and a CH3 domain. In certain embodiments, an Fc chain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc chain (i.e., a hinge domain, a CH2 domain, and a CH3 domain). In certain embodiments, an Fc chain comprises a hinge domain (or portion thereof) fused to a CH3 domain (or portion thereof). In certain embodiments, an Fc chain comprises a CH2 domain (or portion thereof) fused to a CH3 domain (or portion thereof). In certain embodiments, an Fc chain consists of a CH3 domain or portion thereof. In certain embodiments, an Fc chain consists of a hinge domain (or portion thereof) and a CH3 domain (or portion thereof). In certain embodiments, an Fc chain consists of a CH2 domain (or portion thereof) and a CH3 domain. In certain embodiments, an Fc chain consists of a hinge domain (or portion thereof) and a CH2 domain (or portion thereof). In certain embodiments, an Fc chain lacks at least a portion of a CH2 domain (e.g., all or part of a CH2 domain). An Fc chain herein generally refers to a polypeptide comprising all or part of the Fc chain of an immunoglobulin heavy-chain. This includes, but is not limited to, polypeptides comprising the entire CHI, hinge, CH2, and/or CH3 domains as well as fragments of such peptides comprising only, e.g., the hinge, CH2, and CH3 domain. The Fc chain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As with Fc variants and native Fc's, the term Fc chain includes molecules in monomeric or multimeric form, whether digested from whole antibody or produced by other means. In some embodiment, the Fc chain comprises the carboxy-terminal portions of both heavy chains held together by disulfides. In certain embodiments, an Fc chain consists of a CH2 domain and a CH3 domain.

In some embodiments, an Fc polypeptide comprises part or all of a wild-type hinge sequence (generally at its N-terminal). In some embodiments, an Fc polypeptide does not comprise a functional or wild-type hinge sequence.

As used herein, the term “CH1 domain” refers to the first constant domain of an antibody heavy chain (e.g., amino acid positions 118-215 of human IgG1, according to the EU index). The term includes naturally occurring CH1 domains and engineered variants of naturally occurring CH1 domains (e.g., CH1 domains comprising one or more amino acid insertions, deletions, substitutions, or modifications relative to a naturally occurring CH1 domain).

As used herein, the term “CH2 domain” refers to the second constant domain of an antibody heavy chain (e.g., amino acid positions 231-340 of human IgG1, according to the EU index). The term includes naturally occurring CH2 domains and engineered variants of naturally occurring CH2 domains (e.g., CH2 domains comprising one or more amino acid insertions, deletions, substitutions, or modifications relative to a naturally occurring CH2 domain).

As used herein, the term “CH3 domain” refers to the third constant domain of an antibody heavy chain (e.g., amino acid positions 341-447 of human IgG1, according to the EU index). The term includes naturally occurring CH3 domains and engineered variants of naturally occurring CH3 domains (e.g., CH3 domains comprising one or more amino acid insertions, deletions, substitutions, or modifications relative to a naturally occurring CH3 domain).

As used herein, the term “EU index” refers to the EU numbering convention for the Fc domains of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. All numbering of amino acid positions of the Fc polypeptides, or fragments thereof, used herein is according to the EU index.

As used herein, the term “specifically binds,” “specifically binding,” “binding specificity” or “specifically recognized” refers that an antigen binding protein or antigen-binding fragment thereof that exhibits appreciable affinity for an antigen (e.g., a TSHR antigen, e.g., thyroid stimulating hormone (TSH)) and does not exhibit significant cross reactivity to a different target protein. As used herein, the term “affinity” refers to the strength of the interaction between an antigen binding protein or antigen-binding fragment thereof antigen binding site and the epitope to which it binds. Methods to determine such specific binding are also well known in the art. In certain embodiments, the antigen binding protein or antigen binding fragment thereof can bind to a human thyroid stimulating hormone receptor (TSHR), but not to TSHR from other species. Alternatively, in some embodiments, the antigen binding proteins or antigen binding fragments bind to human TSHR and to TSHR from one or more non-human species. In certain exemplary embodiments, affinity is measured by surface plasmon resonance (SPR), e.g., in a Biacore instrument. As readily understood by those skilled in the art, an antigen binding protein affinity may be reported as a dissociation constant (KD) in molarity (M). The antigen binding protein or antigen-binding fragment thereof of the disclosure have KD values in the range of about 10⁻⁵ M to about 10⁻¹² M (i.e., low micromolar to picomolar range), about 10⁻⁷ M to 10⁻¹¹ M, about 10⁻⁸ M to about 10⁻¹⁰ M, about 10⁻⁹ M. In certain embodiments, the antigen binding protein or antigen-binding fragment thereof has a binding affinity of about 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In certain embodiments, the antigen binding protein or antigen-binding fragment thereof has a binding affinity of about 10⁻⁷ M to about 10⁻⁹ M (nanomolar range).

Specific binding can be determined according to any art-recognized means for determining such binding. In some embodiments, specific binding is determined by competitive binding assays (e.g., ELISA) or Biacore assays. In certain embodiments, the assay is conducted at about 20° C., 25° C., 30° C., or 37° C. In certain embodiments, the assay is conducted at physiological pH, at an acidic pH (e.g., a pH more acidic than physiological pH), or at a basic pH (e.g., a pH more basic than physiological pH).

As used herein, “administer” or “administration” refers to the act of injecting or otherwise physically delivering a substance as it exists outside the body (e.g., an isolated binding polypeptide provided herein) into a patient, such as by, but not limited to, subcutaneous, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, mice, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “about” or “approximately” means within about 20%, such as within about 10%, within about 5%, or within about 1% or less of a given value or range.

### TSHR-Related Diseases

As used herein, the terms “thyroid stimulating hormone receptor” or “TSHR” refer to full length human TSHR protein or the proteolytically cleaved TSHR protein made up of two subunits. The full-length TSHR protein comprises an amino acid sequence of 764 amino acids in length. Although it may exist as a single polypeptide chain under some situations on thyroid cells or in extra-thyroid cells, most of the TSHR on thyroid cells is cleaved and divided into two subunits, A and B. A is considered an extracellular subunit and B is a large intracellular portion. The two subunits can be crosslinked by disulfide bonds. To achieve full functionality, TSHR also undergoes N-linked glycosylation, palmitoylation and other types of post-translational modifications. TSHR is a member of the G-protein coupled receptor (GPCR) family and consists of three domains: the extracellular leucine-rich repeat domain (LRD), the hinge region and a transmembrane domain (TMD) with an intracellular C-terminus (Nunez Miguel, et al. (2004) Thyroid 14:991-1011).

In the thyroid, the TSHR is present on the basal membrane of thyroid follicular epithelial cells. Binding of TSH to the TSHR starts activation of the TSHR signaling cascade which involves binding of G-proteins to the TSHR followed by stimulation of the cyclic AMP pathway and synthesis of thyroid hormones, including thyroxine (T4) and triiodothyronine (T3) (Sanders J et al (1997) Balliere's Clinical Endocrinology and Metabolism. Ed T F Davies 11: 451-479; pub Balliere Tindall, London and Latif R et al (2009) Endocrinology and Metabolism Clinics of North America 38: 319-341). In certain embodiments, TSHR is a cell surface receptor that is anchored to the cell membrane. In certain embodiments, a subunit of TSHR may be shed from the cell surface as a soluble protein. In certain embodiments, the subunit of TSHR that is shed from the cell surface is the A subunit.

In some instances, GPCRs are known to be in close physical or functional proximity to other types of cell surface receptors, leading to signaling or “crosstalk” between the GPCRs and other cell surface receptors, for example ion channels (Davies et al. (2023) Prog Mol Biol Transl Sci 195:101-120), tyrosine kinase receptors (Krieger et al. (2020) Pharmacol Ther 209:107502) and integrins (Teoh et al. (2012) Journal of Allergy, vol. 2012, Article ID: 341282). IGF-1R, a tyrosine kinase receptor, and TSHR exhibit crosstalk in periorbital fibroblasts, which can lead to amplification of signaling pathways associated with thyroid eye disease. Teprotumumab, a fully human monoclonal antibody to IGF-1R, attenuates signaling initiated at either TSHR or IGF-1R, thereby blocking pathologic immune responses in active thyroid eye disease (Douglas et al. (2020) N Engl J Med 382:341-52). Thus, functional antagonism of TSHR signaling may have a broader therapeutic potential by impacting functional signaling of other receptors that may be involved in TSHR mediated diseases. In certain embodiments, the antigen binding proteins or the antigen binding fragment thereof as disclosed herein may block or inhibit signaling between TSHR and other cell surface receptors (e.g., IGF-1R). In certain embodiments, the antigen binding proteins or the antigen binding fragment thereof as disclosed herein may enhance or stimulate signaling between TSHR and other cell surface receptors (e.g., IGF-1R).

In some embodiments, the thyroid associated disease, disorder or condition is a thyroid cancer metastases, goiter, multinodular goiter, congenital hypothyroidism, hyperthyroidism, Graves' disease, ophthalmic Graves' disease, neonatal hyperthyroidism, hypothyroidism, thyroid disease, autoimmune thyroid disease, Hashimoto's thyroiditis, Painless thyroiditis (PT), Postpartum thyroiditis (PPT), and Subacute thyroiditis (SAT), Graves' ophthalmopathy and pre-tibial myxoedema, Graves' eye disease, thyroid eye disease (TED) (e.g. Acute mild TED, Acute moderate to severe TED, Acute severe sight threatening TED, Recurrent TED, Chronic TED, TED secondary to treatment with radioactive iodine), Graves' disease (Acute moderate to severe graves' disease, management, Chronic Grave's disease management, Thyrotoxicosis), Graves' disease associated disorders (e.g., thyroid acropathy, Graves dermopathy), thyroid cancer (e.g., differentiated thyroid carcinoma (DTC), recurrent radioactive iodine resistant DTC, metastatic DTC), or follicular thyroid carcinoma. In some embodiments, the follicular thyroid carcinoma is iodine resistant or exhibits reduced iodine uptake. In some embodiments, the thyroid associated disease, disorder or condition is an acute sight threatening thyroid eye disease (Dysthyroid optic neuropathy or DON), acute moderate to severe thyroid eye disease, chronic thyroid eye disease, acute severe thyrotoxicosis, Graves' disease or acute severe Graves' disease.

### Autoimmune Thyroid Diseases

Autoimmune thyroid diseases (AITD) are one of the most prevalent autoimmune conditions. The major thyroid autoantigens targeted by the autoimmune system are thyroid peroxidase (TPO), thyroglobulin (Tg) and TSHR. TPO autoantibodies and thyroglobulin autoantibodies are serological markers of thyroid autoimmunity in different forms of autoimmune thyroid diseases including Hashimoto's thyroiditis, Graves' disease and postpartum thyroiditis (PPT) (Rees Smith B ef al (2007) Thyroid 17: 923-938). TSHR autoantibodies are markers of TSHR autoimmunity, and in particular, Graves' disease. Furthermore, these TSHR autoantibodies are responsible for potentially initiating and driving the pathology of Graves' disease. Types of TSHR autoantibodies may include those commonly referred to as stimulating autoantibodies, blocking autoantibodies and neutral autoantibodies.

Thyroid stimulating autoantibodies bind to the TSHR and mimic the actions of TSH, thereby stimulating the thyroid to produce high levels of T3 and T4 and are thereby considered agonistic antibodies. The feedback control mechanism of thyroid function is no longer effective in the presence of thyroid stimulating autoantibodies and the patients present with clinical symptoms of a hyperactive thyroid characterized by an excess of thyroid hormones in serum and their metabolic consequences. This condition is known as Graves' disease or in some geographies, as Basedow's disease. These TSHR-activating autoantibodies may also interact with TSHR found in the retro-orbital tissue and contribute to the development of eye-related symptoms of Graves' disease, known as Graves' ophthalmopathy, Graves orbitopathy or thyroid eye disease (TED). Other signs and symptoms of Graves' disease include hyperthyroidism, goiter, and pretibial myxedema. Symptoms of hyperthyroidism are mainly insomnia, hand tremors, hyperactivity, hair loss, excessive sweating, oligomenorrhea, itching, heat intolerance, weight loss, diarrhea, frequent defecation, palpitations, periodic partial muscle weakness or paralysis, and skin warmth and moistness.

### Cancer

In some embodiments, the antigen binding protein or the antigen binding fragment thereof that specifically binds to TSHR can be used to treat a cancer in a subject. In some embodiments, the cancer is a thyroid cancer. In some embodiments, the cancer is a thyroid cancer that has disseminated from its primary site in the thyroid to other tissues and organs. In some embodiments, the cancer is an extra-thyroid cancer (e.g., a cancer not associated with the thyroid).

Expression of TSHR has been recognized in benign or malignant thyroid cells (thyrocytes), serving as the receptor for TSH. Activation of the signaling cascade through TSHR has been shown to serve as oncogenic pathways in thyroid cancer. Since the seminal publication by Ichikawa et al ((1976) Journal of Clinical Endocrinology and Metabolism 42:395-398), several independent studies have demonstrated significant continued expression of TSHR in the majority of differentiated thyroid carcinomas (Rowe et al. (2017) Endocr Relat Cancer 24(6):R191-R202). The targeting of TSHR is particularly useful in the context of recurrent or metastatic differentiated thyroid carcinoma. In this setting, antagonistic blockade of TSHR would prevent binding of TSH to TSHR. Suppression of TSH has been demonstrated to aid in reduction of thyroid tumor size and prevention of thyroid cancer recurrence (Mazzaferri et al. (1994) American Journal of Medicine 97:418-428). It is also thought that this would sensitize thyroid cancers to radioiodine exposure, which would be of use in the setting of recurrent differentiated thyroid carcinoma where cancers often develop adaptive resistance to radioiodine therapy. Furthermore, thyroid autoimmunity is associated with increased risk of thyroid cancer, in particular differentiated thyroid cancers.

Thus, clinically useful strategies have been proposed to treat well-differentiated thyroid cancer, which harbors a higher density of TSHR. Targeted TSHR therapies would be of great value in reducing the size of thyroid tumors prior to surgical intervention which requires margins that may include important tissues such as trachea, larynx, nerves, lymphatic vessels, blood vessels and bone. In addition, targeted therapies would be of great value in lengthening survival time or improving overall survival in the setting of unresectable or disseminated thyroid cancers. In settings where thyroid cancer patients have underlying autoimmune thyroid disease, targeted therapeutics may have a role in antagonizing the proliferative effect of autoantibodies or reducing lymphocytic thyroiditis that is associated with thyroid autoantibody positivity (Viola et al. (2023) Endocr Relat Cancer 30(7):e230042). Finally, targeted therapeutics conjugated with radiolabeled isotopes could improve diagnosis and detection of TSHR expressing thyroid tumors. Expression of TSHR in extra-thyroid cancer cells is also documented. For instance, expression of TSHR has been reported in human ovarian tissue (Aghajanova et al. (2009)). Functionality of TSHR in primary human ovarian tissues under TSH or thyroid hormone stimulation was also demonstrated through assessing downstream signaling pathways or metabolites. TSHR activation has been shown to activate canonical G-protein coupled signaling pathways and transregulated activation of epithelial growth factor receptor (EGFR) to promote ovarian cancer cell proliferation (Huang, et al. (2016) Science Reports 6:27471). TSHR has also been shown to be expressed in liver cancer, glioma (low grade and Grade IV glioblastoma), and breast cancer.

### TSHR-Targeting Binding Proteins

In an aspect of the present disclosure, provided herein are antigen binding proteins or antigen binding fragments thereof that bind specifically to thyroid stimulating hormone receptor (TSHR). In some embodiments, the TSHR binding proteins block the binding of TSHR autoantibodies to TSHR.

One component of an antigen binding protein or an antigen binding fragment thereof of the present disclosure is one or more antigen binding domains or binding specificity which binds one or more cell surface targets (e.g., membrane bound TSHR) or one or more soluble targets (e.g., soluble TSHR).

Any type of binding moiety that specifically binds to a specific receptor subunit can be employed in the antigen binding protein or the antigen binding fragment thereof disclosed herein. In certain embodiments, the binding moiety comprises an antibody variable domain. Exemplary binding moieties comprising an antibody variable domain include, without limitation, a VH, a VL, a VHH, a VH/VL pair, an scFv, a diabody, or a Fab. Other suitable binding moiety formats include, without limitation, lipocalins (see e.g., Gebauer M. et al., 2012, Method Enzymol. 503:157-188, which is incorporated by reference herein in its entirety), adnectins (see e.g., Lipovsek D., 2011, Protein Eng. Des. Sel. 24:3-9, which is incorporated by reference herein in its entirety), avimers (see e.g., Silverman J, et al., 2005, Nat. Biotechnol. 23:1556-1561, which is incorporated by reference herein in its entirety), fynomers (see e.g., Schlatter D, et al., 2012, mAbs 4:497-508, which is incorporated by reference herein in its entirety), kunitz domains (see e.g., Hosse R. J. et al., 2006, Protein Sci. 15:14-27, which is incorporated by reference herein in its entirety), knottins (see e.g., Kintzing J. R. et al., 2016, Curr. Opin. Chem. Biol. 34:143-150, which is incorporated by reference herein in its entirety), affibodies (see e.g., Feldwisch J. et al., 2010 J. Mol. Biol. 398:232-247, which is incorporated by reference herein in its entirety), and DARPins (see e.g., Pluckthun A., 2015, Annu. Rev. Pharmacol. Toxicol. 55:489-511, which is incorporated by reference herein in its entirety).

In certain embodiments, the binding domain comprises the heavy and/or light chain variable regions of a conventional antibody or antigen binding fragment thereof (e.g., a Fab or scFv), wherein the term “conventional antibody” is used herein to describe heterotetrameric antibodies containing heavy and light immunoglobulin chains arranged according to the “Y” configuration. Such conventional antibodies may derive from any suitable species including but not limited to antibodies of llama, alpaca, camel, mouse, rat, rabbit, goat, hamster, chicken, monkey, or human origin. In certain exemplary embodiments, the conventional antibody comprises a heavy chain variable domain (VH) and a light chain variable domain (VL) wherein the VH and/or VL domains or one or more complementarity determining regions (CDRs) thereof are derived from the same antibodies. In certain embodiments, the conventional antibody antigen binding region may be referred to as a “Fab” (Fragment antigen-binding). The Fab comprises one constant and one variable domain from each of heavy chain and light chain. The variable heavy and light chains contain the CDRs responsible for antigen binding.

In certain embodiments, the antigen binding protein or the antigen binding fragment thereof of the present disclosure comprises a heavy chain variable (VH) domain and a light chain variable (VL) domain, wherein: a. the VH domain comprises a HCDR1 sequence comprising the amino acid sequence of SEQ ID NO: 1, a HCDR2 sequence comprising the amino acid sequence of SEQ ID NO: 2, and a HCDR3 sequence comprising the amino acid sequence of SEQ ID NO: 3; and the VL domain comprises a LCDR1 sequence comprising the amino acid sequence of SEQ ID NO: 4, a LCDR2 sequence comprising the amino acid sequence of SEQ ID NO: 5, and a LCDR3 sequence comprising the amino acid sequence of SEQ ID NO: 6; b. the VH domain comprises a HCDR1 sequence comprising the amino acid sequence of SEQ ID NO: 11, a HCDR2 sequence comprising the amino acid sequence of SEQ ID NO: 12, and a HCDR3 sequence comprising the amino acid sequence of SEQ ID NO: 13; and the VL domain comprises a LCDR1 sequence comprising the amino acid sequence of SEQ ID NO: 14, a LCDR2 sequence comprising the amino acid sequence of SEQ ID NO: 15, and a LCDR3 sequence comprising the amino acid sequence of SEQ ID NO: 16; c. the VH domain comprises a HCDR1 sequence comprising the amino acid sequence of SEQ ID NO: 21, a HCDR2 sequence comprising the amino acid sequence of SEQ ID NO: 22, and a HCDR3 sequence comprising the amino acid sequence of SEQ ID NO: 23; and the VL domain comprises a LCDR1 sequence comprising the amino acid sequence of SEQ ID NO: 24, a LCDR2 sequence comprising the amino acid sequence of SEQ ID NO: 25, and a LCDR3 sequence comprising the amino acid sequence of SEQ ID NO: 26; d. the VH domain comprises a HCDR1 sequence comprising the amino acid sequence of SEQ ID NO: 31, a HCDR2 sequence comprising the amino acid sequence of SEQ ID NO: 32, and a HCDR3 sequence comprising the amino acid sequence of SEQ ID NO: 33; and the VL domain comprises a LCDR1 sequence comprising the amino acid sequence of SEQ ID NO: 34, a LCDR2 sequence comprising the amino acid sequence of SEQ ID NO: 35, and a LCDR3 sequence comprising the amino acid sequence of SEQ ID NO: 36; or e. the VH domain comprises a HCDR1 sequence comprising the amino acid sequence of SEQ ID NO: 41, a HCDR2 sequence comprising the amino acid sequence of SEQ ID NO: 42, and a HCDR3 sequence comprising the amino acid sequence of SEQ ID NO: 43; and the VL domain comprises a LCDR1 sequence comprising the amino acid sequence of SEQ ID NO: 44, a LCDR2 sequence comprising the amino acid sequence of SEQ ID NO: 45, and a LCDR3 sequence comprising the amino acid sequence of SEQ ID NO: 46.

In certain embodiments,
a. the VH comprises an amino acid sequence at least 90% identical (i.e., 90%, 91%, 92%, 93%, 94%, 95%, 96%, 97%, 98%, 99%, or 100% identical) to SEQ ID NO: 7 and the VL comprises an amino acid sequence at least 90% identical (i.e., 90%, 91%, 92%, 93%, 94%, 95%, 96%, 97%, 98%, 99%, or 100% identical) to SEQ ID NO: 8;
b. the VH comprises an amino acid sequence at least 90% identical (i.e., 90%, 91%, 92%, 93%, 94%, 95%, 96%, 97%, 98%, 99%, or 100% identical) to SEQ ID NO: 17 and the VL comprises an amino acid sequence at least 90% identical (i.e., 90%, 91%, 92%, 93%, 94%, 95%, 96%, 97%, 98%, 99%, or 100% identical) to SEQ ID NO: 18;
c. the VH comprises an amino acid sequence at least 90% identical (i.e., 90%, 91%, 92%, 93%, 94%, 95%, 96%, 97%, 98%, 99%, or 100% identical) to SEQ ID NO: 27 and the VL comprises an amino acid sequence at least 90% identical (i.e., 90%, 91%, 92%, 93%, 94%, 95%, 96%, 97%, 98%, 99%, or 100% identical) to SEQ ID NO: 28;
d. the VH comprises an amino acid sequence at least 90% identical (i.e., 90%, 91%, 92%, 93%, 94%, 95%, 96%, 97%, 98%, 99%, or 100% identical) to SEQ ID NO: 37 and the VL comprises an amino acid sequence at least 90% identical (i.e., 90%, 91%, 92%, 93%, 94%, 95%, 96%, 97%, 98%, 99%, or 100% identical) to SEQ ID NO: 38; or
e. the VH comprises an amino acid sequence at least 90% identical (i.e., 90%, 91%, 92%, 93%, 94%, 95%, 96%, 97%, 98%, 99%, or 100% identical) to SEQ ID NO: 47 and the VL comprises an amino acid sequence at least 90% identical (i.e., 90%, 91%, 92%, 93%, 94%, 95%, 96%, 97%, 98%, 99%, or 100% identical) to SEQ ID NO: 48.

In certain embodiments,
a. the VH comprises an amino acid sequence of SEQ ID NO: 7 and the VL comprises an amino acid sequence of SEQ ID NO: 8;
b. the VH comprises an amino acid sequence of SEQ ID NO: 17 and the VL comprises an amino acid sequence of SEQ ID NO: 18;
c. the VH comprises an amino acid sequence of SEQ ID NO: 27 and the VL comprises an amino acid sequence of SEQ ID NO: 28;
d. the VH comprises an amino acid sequence of SEQ ID NO: 37 and the VL comprises an amino acid sequence of SEQ ID NO: 38; or
e. the VH comprises an amino acid sequence of SEQ ID NO: 47 and the VL comprises an amino acid sequence of SEQ ID NO: 48.

In certain embodiments, the antigen binding protein or the antigen binding fragment thereof comprises:
a. a heavy chain polypeptide comprising an amino acid sequence at least 90% identical to SEQ ID NO: 9, and a light chain polypeptide comprising an amino acid sequence at least 90% identical to SEQ ID NO: 10;
b. a heavy chain polypeptide comprising an amino acid sequence at least 90% identical to SEQ ID NO: 19, and a light chain polypeptide comprising an amino acid sequence at least 90% identical to SEQ ID NO: 20;
c. a heavy chain polypeptide comprising an amino acid sequence at least 90% identical to SEQ ID NO: 29, and a light chain polypeptide comprising an amino acid sequence at least 90% identical to SEQ ID NO: 30;
d. a heavy chain polypeptide comprising an amino acid sequence at least 90% identical to SEQ ID NO: 39, and a light chain polypeptide comprising an amino acid sequence at least 90% identical to SEQ ID NO: 40; or
e. a heavy chain polypeptide comprising an amino acid sequence at least 90% identical to SEQ ID NO: 49, and a light chain polypeptide comprising an amino acid sequence at least 90% identical to SEQ ID NO: 50.

In certain embodiments, the antigen binding protein or the antigen binding fragment thereof comprises:
a. a heavy chain polypeptide comprising SEQ ID NO: 9, and a light chain polypeptide comprising SEQ ID NO: 10;
b. a heavy chain polypeptide comprising SEQ ID NO: 19, and a light chain polypeptide comprising SEQ ID NO: 20;
c. a heavy chain polypeptide comprising SEQ ID NO: 29, and a light chain polypeptide comprising SEQ ID NO: 30;
d. a heavy chain polypeptide comprising SEQ ID NO: 39, and a light chain polypeptide comprising SEQ ID NO: 40; or
e. a heavy chain polypeptide comprising SEQ ID NO: 49, and a light chain polypeptide comprising SEQ ID NO: 50.

In other embodiments, the specific receptor subunit binding subunit comprises at least a CDR or VHH domain of a VHH antibody or Nanobody. VHH antibodies, which are camelid-derived heavy chain antibodies, are composed of two heavy chains and are devoid of light chains (Hamers-Casterman, et al. Nature. 1993; 363; 446-8). Each heavy chain of the VHH antibody has a variable domain at the N-terminus, and these variable domains are referred to in the art as “VHH” domains in order to distinguish them from the variable domains of the heavy chains of the conventional antibodies i.e., the VH domains. Similar to conventional antibodies, the VHH domains of the molecule comprise HCDR1, HCDR2 and HCDR3 regions which confer antigen binding specificity and therefore VHH antibodies or fragments such as isolated VHH domains, are suitable as components of the multispecific binding proteins of the present disclosure.

In some embodiments, the TSHR-binding protein is a multispecific antigen binding protein. The term “multispecific antigen binding protein” as used herein refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. In certain embodiment, the multispecific antigen binding molecules of the disclosure comprises at least a first binding specificity for a subunit of a receptor and at least a second binding specificity for a subunit. A multispecific antigen-binding molecule can be a single multifunctional polypeptide, or it can be a multimeric complex of two or more polypeptides that are covalently or non-covalently associated with one another. The term “multispecific antigen-binding molecules” includes antibodies of the present disclosure that may be linked to or co-expressed with another functional molecule, e.g., another antibody, antibody fragment, peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In certain exemplary embodiments, an antibody of the present disclosure is functionally linked to another antibody or antigen-binding fragment thereof to produce a bispecific antibody with a second binding specificity.

Methods for making multispecific binding proteins are well known. For example, traditionally, the recombinant production of bispecific antibodies was based on the co-expression of two immunoglobulin heavy chain/light chain pairs, where the two heavy chains have different specificities (Milstein et al., Nature 305:537 (1983)). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. More modern techniques for generating bispecific antibodies employ heterodimerization domains that favor desired pairing of heavy chain from the antibody with a first specificity to the heavy chain of an antibody with a second specificity.

Antibody variable domains with the desired binding specificities can be fused to immunoglobulin constant domain sequences. The fusion typically is with an immunoglobulin heavy chain constant domain, comprising at least part of the hinge, CH2, and CH3 regions. It may have the first heavy chain Fc domain (CH1) containing the site necessary for light chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. Enzymol. 121:210 (1986).

### CD80 Binding

The antigen binding proteins of the disclosure possess reduced binding to the protein cluster of differentiation 80 (CD80), also known as B7-1. CD80 is a co-stimulatory molecule found on numerous immune cells, including antigen-presenting cells. CD80 serves as a ligand for CD28 and CTLA-4 on the surface of T cells. Interaction of CD80 with CD28 triggers costimulatory signals and results in enhanced and sustained T-cell activation. Interaction of CD80 with CTLA-4 inhibits parts of T-cell effector function. Given the important role CD80 plays in regulating the immune response, off-target binding to CD80 by an antigen binding protein of the disclosure may have a deleterious effect on a subject. The TSHR parental antibody described here possessed detectable off-target binding to CD80. Accordingly, the antigen binding proteins of the disclosure have been selected for based, in part, on reduced CD80 binding relative to the parental antibody.

In certain embodiments, the antigen binding protein or antigen binding fragment thereof has reduced binding to CD80 compared to an antigen binding protein comprising a VH domain of SEQ ID NO: 61 and a VL domain of SEQ ID NO: 62.

In certain embodiments, the antigen binding protein or antigen binding fragment thereof has at least about 2-fold, at least about 3-fold, at least about 4-fold, or at least about 5-fold reduced binding to CD80 compared to an antigen binding protein comprising a VH domain of SEQ ID NO: 61 and a VL domain of SEQ ID NO: 62.

In certain embodiments, binding to CD80 is determined by detecting binding of the antigen binding protein or the antigen binding fragment thereof to a cell that overexpresses CD80. In certain embodiments, the antigen binding protein or the antigen binding fragment thereof is incubated with the cell that overexpresses CD80 at a concentration of 100 μg/mL. In certain embodiments, binding is detecting by flow cytometry.

### Effector Function Mutations

As discussed above, the antigen binding proteins of the disclosure can be provided in various isotypes and with different Fc domains. The Fc region of the antigen binding protein primarily determines its effector function in terms of Fc binding, antibody-dependent cell-mediated cytotoxicity (ADCC) activity, complement dependent cytotoxicity (CDC) activity, and antibody-dependent cell phagocytosis (ADCP) activity. These “cellular effector functions”, as distinct from effector T cell function, involve the recruitment of cells bearing Fc receptors to the site of the target cells, resulting in killing of the antibody-bound cell.

An antigen binding protein or an antigen binding fragment thereof according to the present invention may be one that exhibits reduced effector function. In certain embodiments, the one or more mutations reduces one or more of antibody dependent cellular cytotoxicity (ADCC), antibody dependent cellular phagocytosis (ADCP), or complement dependent cytotoxicity (CDC). In certain embodiments, an antibody according to the present invention may lack ADCC, ADCP, and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

In certain embodiments, the first and the second Fc domain comprises one or more mutations that reduces Fc effector function. In certain embodiments, the first Fc domain and the second Fc domain each comprise a L234A and L235A mutation. These IgG1 mutations are also known as the “LALA” mutations and are described in further detail in Xu et al. (Cell Immunol. 2000; 200:16-26). In certain embodiments, the first Fc domain and the second Fc domain each comprise a L234A, L235A, G237A, and/or P329G mutations. The Fc domain amino acid positions referred to herein are based on EU antibody numbering. Alternatively, an antibody may have a Fc domain which is effector null. An antibody may have a heavy chain Fc domain that does not bind Fcγ receptors, for example the Fc domain may comprise a L235E mutation. Another optional mutation for a heavy chain Fc domain is S228P, which increases stability. A heavy chain Fc domain may be an IgG4 comprising both the L235E mutation and the S228P mutation. This “IgG4-PE” heavy chain Fc domain is effector null. A disabled IgG1 heavy chain Fc domain may contain alanine at position 234, 235, and/or 237 (EU index numbering), e.g., it may be an IgG1 sequence comprising the L234A, L235A, and/or G237A mutations (“LALAGA”).

Human IgG1 Fc domains containing specific mutations or altered glycosylation on residue Asn297 (e.g., N297Q, N297D, and N297K, according to EU index numbering) have been shown to reduce binding to Fc receptors.

In other embodiments, it may be desirable to enhance the binding of the Fc region of an antigen binding protein to human Fc gamma receptor IIIA (FcγRIIIA) relative to that of the Fc region of a corresponding naturally occurring antibody. In certain embodiments, a Fc domain may be engineered for enhanced ADCC and/or CDC and/or ADCP. The potency of Fc-mediated effects may be enhanced by engineering the Fc domain by various established techniques. Such methods increase the affinity for certain Fc-receptors, thus creating potential diverse profiles of activation enhancement. This can be achieved by modification of one or several amino acid residues. Example mutations are one or more of the residues selected from 239, 332 and 330 for human IgG1 Fc domains (or the equivalent positions in other IgG isotypes). An antibody may thus comprise a human IgG1 Fc domain having one or more mutations independently selected from S239D, I332E and A330L (EU index numbering).

Increased affinity for Fc receptors can also be achieved by altering the natural glycosylation profile of the Fc domain by, for example, generating under fucosylated or de-fucosylated variants. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIA binding capacity. For example, to increase ADCC, residues in the hinge region can be altered to increase binding to FcγRIIIA. Thus, an antibody may comprise a human IgG heavy chain Fc domain that is a variant of a wild-type human IgG heavy chain Fc domain. In certain embodiments, the variant human IgG heavy chain Fc domain binds to human Fcγ receptors selected from the group consisting of FcγRIIB and FcγRIIA with higher affinity than the wild type human IgG heavy chain Fc domain binds to the human FcγRIIIA. The antibody may comprise a human IgG heavy chain Fc domain that is a variant of a wild type human IgG heavy chain Fc domain, wherein the variant human IgG heavy chain Fc domain binds to human FcγRIIIB with higher affinity than the wild type human IgG heavy chain Fc domain binds to human FcγRIIB. The variant human IgG heavy chain Fc domain can be a variant human IgG1, a variant human IgG2, or a variant human IgG4 heavy chain Fc domain. In one embodiment, the variant human IgG heavy chain Fc domain comprises one or more amino acid mutations selected from G236D, P238D, S239D, S267E, L328F, and L328E (EU index numbering system). in another embodiment, the variant human IgG heavy chain Fc domain comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system).

The enhancement of CDC may be achieved by amino acid changes that increase affinity for C1q, the first component of the classic complement activation cascade. Another approach is to create a chimeric Fc domain created from human IgG1 and human IgG3 segments that exploit the higher affinity of IgG3 for C1q. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a Fc domain comprising one or more mutations selected from E345K, E430G, R344D and D356R, in particular a double mutation comprising R344D and D356R (EU index numbering system).

The functional properties of the antigen binding proteins may be further tuned by combining amino acid substitutions that alter Fc binding affinity with amino acid substitutions that affect binding to FcRn. Binding proteins with amino acid substitutions that affect binding to FcRn (also referred to herein as “FcRn variants”) may in certain situations also increase serum half-life in vivo as compared to an unmodified binding protein. As it will be appreciated, any combination of Fc and FcRn variants may be used to tune clearance of the antigen-antibody complex. Suitable FcRn variants that may be combined with any of the Fc variants described herein that include without limitation N434A, N434S, M428L, V308F, V259I, M428L/N434S, V259I/V308F, Y436I/M428L, Y436I/N434S, Y436V/N434S, Y436V/M428L, M252Y, M252Y/S254T/T256E, and V259I/V308F/M428L.

In certain embodiments, one or both of the first Fc heavy chain and second Fc heavy chain comprises one or more substitutions at amino acid positions 252, 254, and/or 256, according to EU numbering. In certain embodiments, the substitution at amino acid position 252 is a tyrosine (Y), the substitution at amino acid position 254 is a threonine (T), and the substitution at amino acid position 256 is a glutamic acid (E).

### Antibody Conjugates

In certain embodiments, the antigen binding protein or the antigen binding fragment thereof that specifically binds TSHR of the present disclosure can be further conjugated to a payload. In certain embodiments, the payload comprises a small molecule. In certain embodiments, the payload comprises a protein or a peptide. In certain embodiments, the payload comprises a polynucleotide molecule. In certain embodiments, the payload comprises a radioisotope. In certain embodiments, the payload comprises a detectable label.

In certain embodiments, the antigen binding protein or antigen binding fragment thereof of the present disclosure is linked to a payload via a linker.

In certain embodiments, the payload can be conjugated to a Fc region of the antibody (e.g., carbohydrate moieties in the Fc region of an antibody can be used to conjugate a therapeutic agent). In certain embodiments, the payload is conjugated to a variable region of the antibody. The engineered carbohydrate moiety is then used to attach a payload. In addition, those of skill in the art will recognize numerous possible variations of the conjugation methods. For example, the carbohydrate moiety can be used to attach polyethyleneglycol in order to extend the half-life of an intact antibody, or antigen-binding fragment thereof, in blood, lymph, or other extracellular fluids. Moreover, it is possible to construct a “divalent conjugate” by attaching therapeutic agents to a carbohydrate moiety and to a free sulfhydryl group. Such a free sulfhydryl group may be located in the hinge region of the antibody component. In some embodiments, the payload can be conjugated to the Fab region of an antibody disclosed herein. In some embodiments, the payload is conjugated to a variable region, for example, of a light chain and/or a heavy chain. In some embodiments, the payload is conjugated to a constant domain, of a light chain and/or a heavy chain.

In certain embodiments, provided herein are antibody-drug conjugates (ADCs). ADCs comprise an antibody conjugated, i.e., covalently attached by a linker, to a drug moiety. The ADCs of the present disclosure may selectively deliver an effective dose of an agent to a tissue whereby greater selectivity, i.e., a lower efficacious dose may be achieved. In some embodiments, the bioavailability of the ADC, or an intracellular metabolite of the ADC, is improved in a subject when compared to the corresponding drug moiety.

In certain embodiments, the drug moiety of the ADC is not cleaved from the antibody until the ADC binds to a cell-surface receptor, i.e., TSHR, or enters a cell with a cell surface receptor specific for the antibody of the ADC. Alternatively, the drug moiety may be cleaved from the antibody after the ADC enters the cell. The drug moiety may be intracellularly cleaved in a subject from the antibody of the compound, or an intracellular metabolite of the compound, by enzymatic action, hydrolysis, oxidation, or other mechanisms.

In certain embodiments, provided herein are radioconjugates, i.e., a radioisotope conjugated with an antigen binding protein or antigen binding fragment thereof of the present disclosure. The radioisotope can serve as a therapeutic agent. The radioisotope can serve as an imaging molecule. Provided herein, in one aspect is a radiotherapeutic that comprises an antibody disclosed herein conjugated with a radioisotope. As used herein, a “radioisotope” and “radionuclide” may be used interchangeably, and may be an alpha particle emitting isotope, a beta particle emitting isotope, and/or a gamma-emitting isotope. Accordingly, an antibody disclosed herein can be conjugated with a beta particle emitter, an alpha particle emitter, and/or a gamma ray emitter. Non-limiting examples of radioisotopes that may be used include the following: 131I, 125I, 123I, 90Y, 177Lu, 186Re, 188Re, 89Sr, 153Sm, 32P, 225Ac, 213Bi, 213Po, 211At, 212Bi, 213Bi, 223Ra, 227Th, 149Tb, 161Tb, 47Sc, 67Cu, 134Ce, 137Cs, 212Pb, and 103Pd. Methods for affixing a radionuclide to an antibody or antibody fragment (i.e., “labeling” the targeting agent such an antibody with a radioisotope) are well known in the art. Specific methods for labeling are described, for example, in U.S. Pat. Nos. 9,603,954, 10,420,851, International Pub. No. WO 2017/155937 and U.S. Provisional Patent Application No. 63/042,651 filed Dec. 9, 2019 and titled “Compositions and methods for preparation of site-specific radioconjugates,” each of which is incorporated by reference herein.

In some embodiments, the radioisotope is selected from the group consisting of Iodine-125, Iodine-123, Iodine-126, Iodine-131, Iodine-133, Bromine-77, Indium-111, Indium-113m, Gallium-67, Gallium-68, Ruthenium-95, Ruthenium-97, Ruthenium-103, Ruthenium-105, Mercury-197, Mercury-203, Rhenium-99m, Rhenium-105, Rhenium-101, Tellurium-121m, Tellurium-122m, Tellurium-125m, Thulium-165, Thulium-167, Thulium-168, Technetium-99m, Fluorine-18, Rhenium-186, Rhenium-188, Silver-Ill, Platinum-197, Palladium-109, Copper-67, Phosphorus-32, Phosphorus-33, Yttrium-90, Scandium-47, Samarium-153, Lutetium-177, Rhodium-105, Praseodymium-142, Praseodymium-143, Terbium-161, Holmium-166 and Gold-199.

### Chimeric Antigen Receptors and T-Cell Receptors

In certain embodiments, the binding domain of the antigen binding protein or the antigen binding fragment thereof that recognizes and binds to TSHR of the present disclosure may be incorporated into a chimeric antigen receptor (CAR) that is to be expressed by an immune cell (e.g., T-cell, NK cell). In certain embodiments, the binding domain is a scFv that is incorporated into a CAR. First generation CARs typically had the intracellular domain from the CD3ζ chain, which is the primary transmitter of signals from endogenous TCRs. Second-generation CARs possess additional intracellular signaling domains from various costimulatory protein receptors (e.g., CD28, 4-1BB, ICOS, etc.) to the cytoplasmic tail of the CAR in order to provide additional signals to the T-cell. Third generation CARs combine multiple signaling domains in order to further augment potency.

CARs are able to redirect immune cell specificity and reactivity toward a selected target exploiting the ligand-binding domain properties. Immune cells, e.g., T-cells, expressing CARs provide a way of treating various cancers. More recently, CAR-T cells have shown therapeutic promise as a treatment for autoimmune diseases.

In certain embodiments, the binding domain of the antigen binding protein or the antigen binding fragment thereof that recognizes and binds to TSHR of the present disclosure may be incorporated into a T-cell receptor (TCR). TCRs are able to interact with immunogenic peptides (epitopes) bound to major histocompatibility complex (MHC) molecules and presented on the surface of target cells. Specific binding of a TCR triggers a signal cascade inside the T cell leading to the proliferation and differentiation into a maturated effector T cell. Endogenous TCRs are diverse in order to be able to target a vast variety of antigens, and this diversity is obtained by genetic rearrangement of different discontinuous segments of genes which code for the different structure regions of TCRs. TCRs are composed of one α chain and one β chain or of one δ chain and one γ chain. The α/β TCR chains are composed of an N-terminal highly polymorphic variable region involved in antigen recognition and an invariant constant region. Exogenous TCRs can be engineered to specifically target an antigen of interest by replacing the variable region of a TCR with an antigen binding domain of an antigen binding protein or antibody.

### Expression of Antigen Binding Proteins

In one aspect, polynucleotides encoding the binding proteins (e.g., antigen binding proteins and antigen binding fragments thereof that specifically bind TSHR) disclosed herein are provided. Methods of making binding proteins comprising expressing these polynucleotides are also provided.

Polynucleotides encoding the TSHR binding proteins disclosed herein are typically inserted in an expression vector for introduction into host cells that may be used to produce the desired quantity of the binding proteins. Accordingly, in certain aspects, the disclosure provides expressions vectors comprising polynucleotides disclosed herein and host cells comprising these vectors and polynucleotides.

The term “vector” or “expression vector” is used herein to mean vectors used in accordance with the present disclosure as a vehicle for introducing into an expressing a desired gene in a cell. As known to those skilled in the art, such vectors may readily be selected from the group consisting of plasmids, phages, viruses and retroviruses. In general, vectors compatible with the disclosure will comprise a selection marker, appropriate restriction sites to facilitate cloning of the desired gene and the ability to enter and/or replicate in eukaryotic or prokaryotic cells.

Numerous expression vector systems may be employed for the purposes of this disclosure. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain Fc domain genes (e.g., human Fc domain genes) synthesized as discussed above.

In other embodiments, the binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding a binding protein, e.g. an antibody or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, from supernatant of lysed cells culture, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, GS-CHO and CHO-K1 (Chinese Hamster Ovary lines), DG44 and DUXB11 (Chinese Hamster Ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HEK (human kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT® cells) (Biowa, Princeton, N.J.)). In one embodiment, NS0 cells may be used. CHO cells are particularly useful. Host cell lines are typically available from commercial services, e.g., the American Tissue Culture Collection, or from authors of published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the binding proteins featured in the disclosure can also be expressed in non-mammalian cells such as bacteria or yeast or plant cells. In this regard, it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the binding proteins can become part of inclusion bodies. In some embodiments, the binding proteins are then isolated, purified and assembled into functional molecules. In some embodiments, the binding proteins of the disclosure are expressed in a bacterial host cell. In some embodiments, the bacterial host cell is transformed with an expression vector comprising a nucleic acid molecule encoding a binding protein of the disclosure.

In addition to prokaryotes, eukaryotic microbes may also be used. Saccharomyces cerevisiae, or common baker's yeast, is the most commonly used among eukaryotic microbes, although a number of other strains are commonly available. For expression in Saccharomyces, the plasmid Yrp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### Formulations/Pharmaceutical Compositions

In certain embodiments, a pharmaceutical composition comprising a pharmaceutically acceptable carrier and a therapeutically effective amount of an antigen-binding protein described herein is provided. Some embodiments include pharmaceutical compositions comprising a therapeutically effective amount of any one of the binding proteins as described herein, in admixture with a pharmaceutically or physiologically acceptable formulation agent selected for suitability with the mode of administration.

Acceptable formulation materials are typically non-toxic to recipients at the dosages and concentrations employed.

In some embodiments, the pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides, e.g., sodium or potassium chloride, or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

In some embodiments the optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

In some embodiments the primary vehicle or carrier in a pharmaceutical composition can be either aqueous or non-aqueous in nature. For example, a suitable vehicle or carrier for injection (e.g., subcutaneous) can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute.

In some embodiments, the pharmaceutical composition for subcutaneous administration can be aqueous in nature. In some embodiments, the pharmaceutical composition for subcutaneous administration can be non-aqueous in nature.

In some embodiments, the pharmaceutical compositions of the disclosure can be selected for parenteral delivery or subcutaneous delivery.

The disclosure also encompasses kits for producing a single dose administration unit. The kits can each contain both a first container having a dried antigen binding protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

The route of administration of the pharmaceutical composition is in accord with known methods, e.g., orally; through injection by subcutaneous, intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

### Method of Treatment/Use

Another aspect of the disclosure is an antigen binding protein or an antigen binding fragment thereof that specifically binds to TSHR as described herein for use as a medicament.

In a particular embodiment, a method of treating a disease or disorder through antagonistic activity is provided, the method comprising administering to a subject in need thereof an effective amount of an antigen binding protein or an antigen binding fragment thereof as described herein. In certain embodiments, a method of treating a disease or disorder through the blocking activity of the antigen binding protein or the antigen binding fragment thereof that specifically binds to TSHR is provided herein. In certain embodiments, the antigen binding protein or the antigen binding fragment thereof that specifically binds to TSHR blocks agonistic TSHR autoantibodies. In certain embodiments, the antigen binding protein or the antigen binding fragment thereof that specifically binds to TSHR may block natural ligands of TSHR. In certain embodiments, the antigen binding protein may not block natural ligands of TSHR. In certain embodiments, the natural ligand of TSHR is thyroid stimulating hormone (TSH).

The antigen binding proteins can be employed in any known assay method, such as competitive binding assays, direct and indirect sandwich assays, and immunoprecipitation assays for the detection and quantitation of one or more target antigens. The binding proteins will bind the one or more target antigens with an affinity that is appropriate for the assay method being employed.

For diagnostic applications, in some embodiments, antigen binding proteins can be labeled with a detectable moiety. The detectable moiety can be any one that is capable of producing, either directly or indirectly, a detectable signal. For example, the detectable moiety can be a radioisotope, such as ³H, ¹⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase.

The antigen binding proteins are also useful for in vivo imaging. An antigen binding protein labeled with a detectable moiety can be administered to an animal, e.g., into the bloodstream, and the presence and location of the labeled antibody in the host assayed. The binding protein can be labeled with any moiety that is detectable in an animal, whether by nuclear magnetic resonance, radiology, or other detection means known in the art.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper that can be pierced by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the present disclosure relates to a method of preventing and/or treating a disease or disorder (e.g., thyroid-related diseases, cancer). In some embodiments, the method comprises administering to a patient a therapeutically effective amount of at least one of the binding proteins, or pharmaceutical compositions related thereto, described herein. In some embodiments, the patient is a human.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use methods and compositions featured in the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of anti-TSHR Antibodies

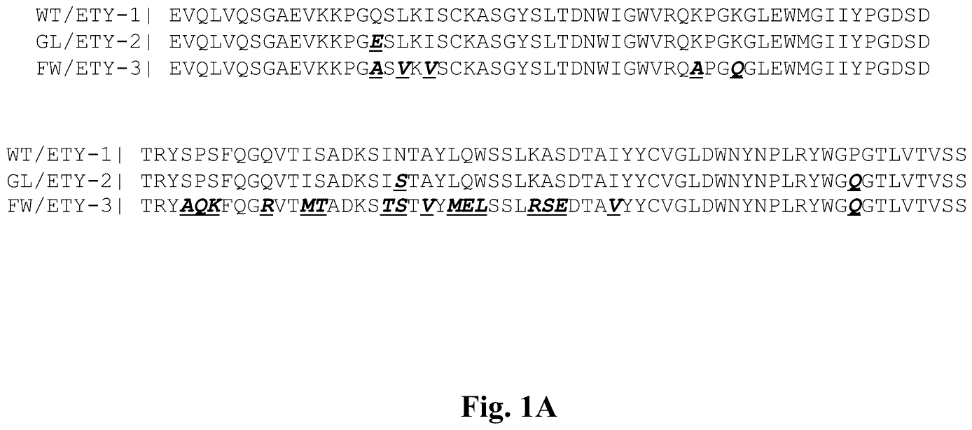

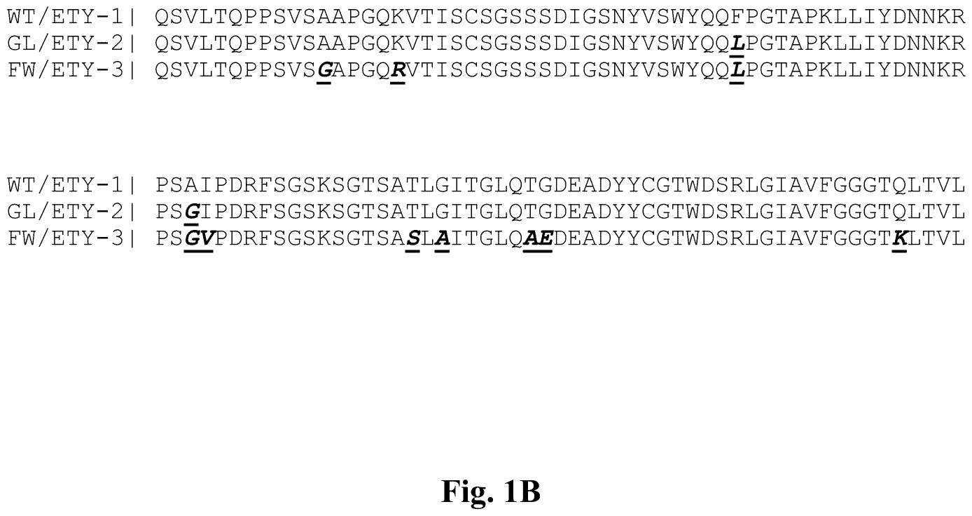

Antibody variants were generated based on the sequence of a parental TSHR-binding antibody (SEQ ID NOs: 1 and 2) (Nunez et al. (2022) J Mol Endocrinol 70(1):e220120 and Sanders et al. (2011) J Mole Endocrinol 46(2):81-99). The variants were obtained from three different experimental pools of 1) a phage display library, 2) a yeast display library, and 3) a rationally designed library. The variants in the pools were either germlined relative to the parental sequence or framework swapped. An alignment of the parental VH and VL (ETY-1) against the germlined (ETY-2) and framework swapped (ETY-3) variants is provided in FIG. 1A (VH alignment) and FIG. 1B (VL alignment).

In total, 169 variants were analyzed by size-exclusion chromatography (SEC), surface plasmon resonance (SPR) for binding affinity, CD80 binding, off-target binding ELISA, Tm/Tagg, and affinity-capture self-interaction nanoparticle spectroscopy (AC-SINS). From this initial characterization, the original list of 169 variants was reduced to 50 variants with a shift of <10 nm in AC-SINS, followed by affinity and sequence diversity.

The subsequent 50 variants were further analyzed for activity (agonist-blocking and independent agonist activity), confirmatory affinity, cell binding activity, and hydrophobic interaction chromatography (HIC). Of these 50 variants, all but two variants showed affinity within four-fold. All but one variant showed activity within three-fold, which is within the noise of the assay.

Of the variants on the germline ETY-2 backbone, 5 variants exhibited better affinity than the parental antibody, designated ETY-67, ETY-77, ETY-78, ETY-79, and ETY-86. Each of the variants contained LCDR1 mutations relative to the parental sequence. Of the variants on the framework swapped (ETY-3) backbone, 5 variants exhibited an affinity within 2-fold of the parental antibody, designated ETY-9, ETY-88, ETY-94, ETY-98, and ETY-155.

This final characterization led to 5 variant antibodies, designated ETY-77, ETY-86, ETY-94, ETY-98, and ETY-155.

The overall characterization of the 169 variants required an affinity that was within two-fold of the parental sequence, an activity that was similar to the parental sequence, and at least 2-fold reduced CD80 binding (average MFI of population at 100 μg/mL) relative to the parental sequence. The final 5 variant antibodies each possess these features.

### Example 2. Determination of the Binding Affinity of TSHR Antibodies

Affinity of the 5 TSHR antibodies to their target antigen was investigated using surface plasmon resonance (SPR), Table 1. Measurements were taken on a Bruker MASS-2 on a High-Capacity Amine (HCA) Sensor (Bruker #1862614) immobilized with ≥10,000 RU anti-HuFc IgG1 (MP Bio #0855071) on spots AB. 40 nM of purified test antibody was injected for 45 sec at 25 μL/min, aiming for a capture level of 400 RU. Biotinylated TSHR22-260 Avi-His tag was injected over spots AB for 4 min at 50 μL/min, followed by 30 min dissociation phase. Analyte concentrations of 6.25 nM, 12.5 nM, 25 nM, 50 nM, and 100 nM were injected in MCK-assay format, from lowest to highest. Running buffer was PBS, pH 7.4 with 0.05% v/v Tween 20. Chip was regenerated with 10 mM Glycine-HCl, pH 1.7 injected for 15 seconds at 25 μL/min. Buffer blank injections and reference spot (A) analyte injections are each subtracted to produce double-subtracted sensorgrams. Data was analyzed using Bruker Analyzer 4 Software

Affinity for TSHR by the antibodies can be screened using a kinetic exclusion assay (KinExA). K_{D} determinations are most accurate when the concentration of the receptor (e.g., TSHR) in the sample is near or below the K_{D}. Binding curves can also be generated for the antibodies.

TABLE 1
Binding Characteristics

| Antibody | kon (M − 1 s − 1) | koff (s − 1) | KD (M) |
|---|---|---|---|
| parental | 9.25E+04 | 5.32E−05 | 5.84E−10 |
| ETY-77 | 1.21E+05 | 3.56E−05 | 2.95E−10 |
| ETY-86 | 1.15E+05 | 3.05E−05 | 2.66E−10 |
| ETY-94 | 7.96E+04 | 6.27E−05 | 7.87E−10 |
| ETY-98 | 9.97E+04 | 6.20E−05 | 6.21E−10 |
| ETY-155 | 7.21E+04 | 4.91E−05 | 6.81E−10 |
| hIgG1 isotype control | — | — | — |
| blank | — | — | — |

### Example 3. Determination of CD80 Binding of TSHR Antibodies Via Flow Cytometry

Cluster of differentiation 80 (CD80), also known as B7-1, is a co-stimulatory molecule found on numerous immune cells, including antigen-presenting cells. CD80 serves as a ligand for CD28 and CTLA-4 on the surface of T cells. Interaction of CD80 with CD28 triggers costimulatory signals and results in enhanced and sustained T-cell activation. Interaction of CD80 with CTLA-4 inhibits parts of T-cell effector function. Given the important role CD80 plays in regulating the immune response, off-target binding to CD80 by an antigen binding protein of the disclosure may have a deleterious effect on a subject. It was observed that the TSHR parental antibody possessed detectable off-target binding to CD80. Accordingly, CD80 off-target binding was one of the parameters for selecting a TSHR antibody from the pool of 169 variants.

CD80 binding was determined using flow cytometry with CHO cells transiently overexpressing CD80 and compared against wild-type CHO cells or with HEK cells overexpressing CD80 and compared against wild-type HEK cells. Two separate isotype controls were also used. CHO WT cells were grown in Ham's F-12K media with 10% FCS, 1% Pen-Strep at 37° C., 5% CO₂. CHO-CD80 cells were grown in Ham's F-12K media with 10% FCS, 1% Pen-Strep, and Puromycin at 37° C., 5% CO₂. HEK WT and HEK CD80 cells were grown in DMEM medium supplemented with 10% FCS, 1% Pen-Strep and 0.25 mg/mL G418 (Geneticin) at 37° C., 5% CO₂. Cells were detached using Accutase (eBioscience) for 5 minutes at 37° C., 5% CO₂. 0.1×10⁵ cells/sample (well) were incubated in PBS supplemented with 2% FCS (FACS buffer) for 15 minutes at 4° C. 1.5 μg/mL, 5 μg/mL, 20 μg/mL and 100 μg/mL of primary antibodies were diluted in FACS buffer and added to the CD80 overexpressing cells and incubated for 15 minutes at 4° C. 100 μg/mL of primary antibody were diluted in FACS buffer and added to the WT cells and incubated for 15 minutes at 4° C. Cells were 2× washed in FACS buffer. Secondary goat anti-human IgG (H+L)—Alexa Fluor 647 antibody (ThermoFisher #A21445, 1/1000 dilution) was added to samples and incubated 15 minutes at 4° C. Samples were washed 2× in FACS buffer. Samples were acquired on a Navios flow cytometer (Beckman Coulter) and data were analyzed using FlowJo software (BD Biosciences).

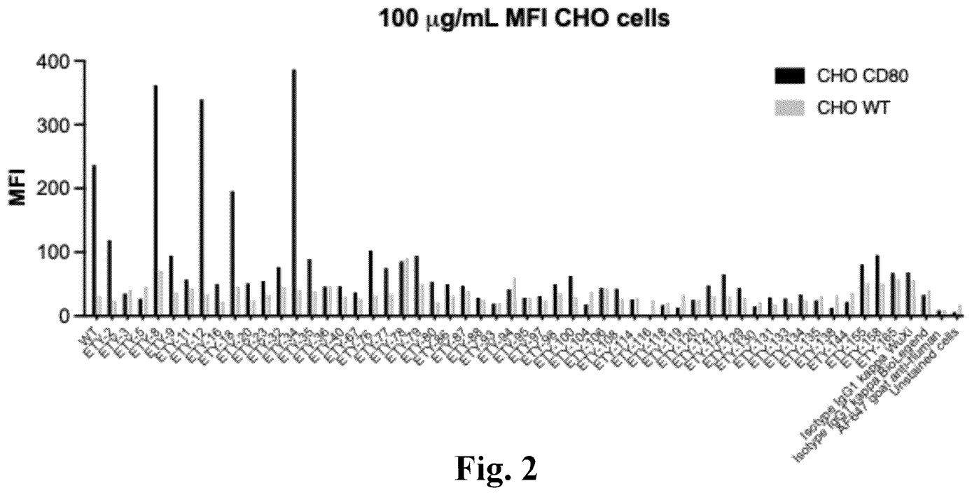

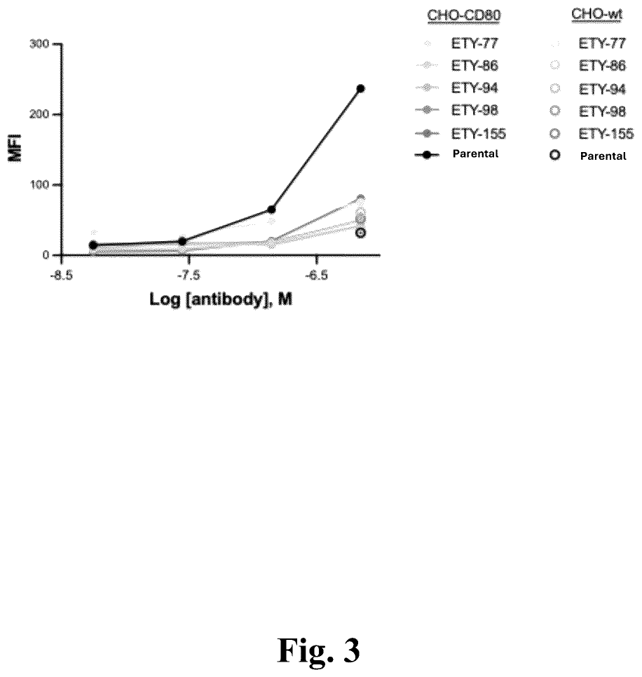

The results for select antibody variants were plotted based on mean fluorescent intensity (MFI) and are shown in FIG. 2 for the assay in CHO cells and are shown in FIG. 3 for the assay in HEK cells. As demonstrated in FIG. 2 and FIG. 3, the 5 variant antibodies, designated ETY-77, ETY-86, ETY-94, ETY-98, and ETY-155, possessed markedly reduced binding to CD80 compared to the parental antibody. Moreover, antibody variants having a single amino acid change relative to the ETY-2 antibody and only five amino acid changes relative to the parental antibody had markedly higher binding to CD80 (see, ETY-8, ETY-12, and ETY-34), demonstrating the unpredictability of amino acid changes needed to reduce CD80 binding.

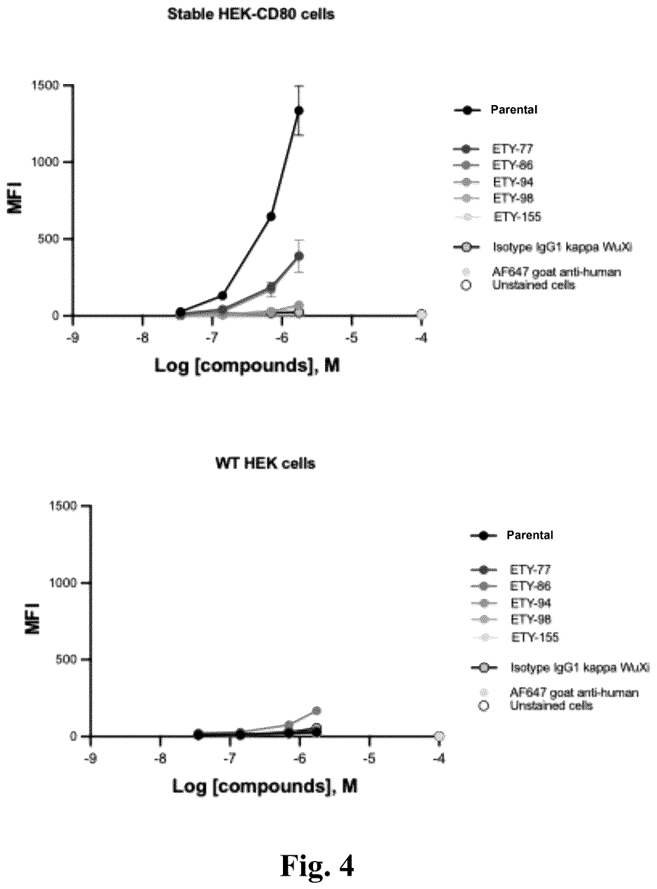

Additionally, the same experiment was repeated with stably expressing HEK-CD80 cells (Kyinno Biotechnology). HEK-CD80 cell line was grown in DMEM with 10% FCS, 1% Pen-Strep, and 0.5 μg/mL Puromycin at 37° C., 5% CO₂. HEK WT cell line was grown in DMEM with 10% FCS, 1% Pen-Strep at 37° C., 5% CO₂. Cells were detached using Accutase (eBioscience) for 5 min at 37° C., 5% CO₂. 0.1×10⁵ cells/sample (well) were incubated in PBS supplemented with 2% FCS (FACS buffer) for 15 min at 4° C. 5 μg/mL, 20 μg/mL, 100 μg/mL and 250 μg/mL of primary antibodies were diluted in FACS buffer and added to the cells and incubated for 20 min at 4° C. Cells were 2× washed in FACS buffer. Secondary goat anti-human IgG (H+L)—Alexa Fluor 647 antibody (ThermoFisher #A21445, 1/2000 dilution) was added to samples and incubated 15 min at 4° C. Samples were then washed 2× in FACS buffer and stained for DAPI. Samples were acquired on a Navios flow cytometer (Beckman Coulter) and data were analyzed using FlowJo software (BD Biosciences). The mean fluorescence intensity of cells is plotted. Results are shown in FIG. 4.

### Determining CD80 Binding of TSHR Antibodies Via DELFIA.

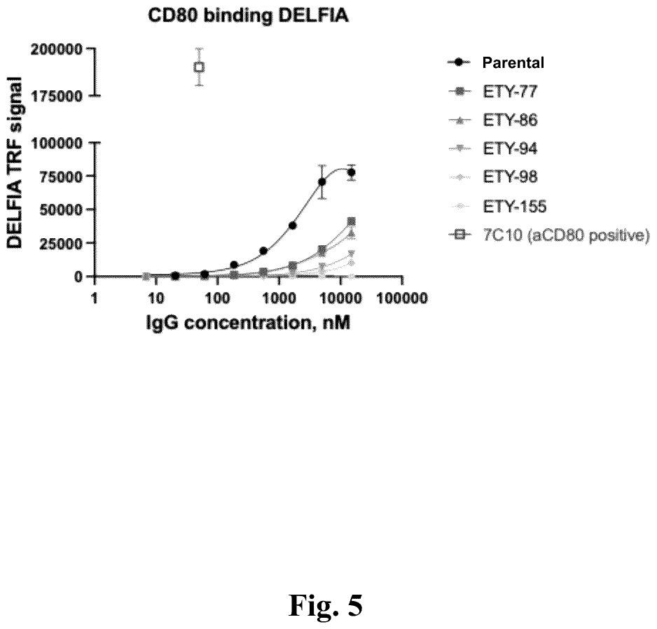

Binding to CD80 was also assessed using recombinant protein in a DELFIA (dissociation-enhanced lanthanide fluorescent immunoassay) format. A standard 96 well plate was coated with streptavidin, 50 μl at 5 μg/ml, 4° C. overnight. The plate was blocked with 3% powdered milk in DPBS at room temperature for 1 hour. The antigen, biotinylated CD80 (Acro Biosystems) at 5 μg/μL, was captured at room temperature for 1 hour. Primary antibody, parental antibody, and variant antibodies were incubated at 15 μM diluted 3-fold 7 points to 20 nM. 7C10 (anti-CD80 positive control) was incubated at a single concentration of 50 nM. All antibodies were incubated at room temperature for 1 hour. Eu3+ mouse anti-human IgG secondary antibody was applied to the assay plate at room temperature for 1 hour. DELFIA enhancement solution was applied to assay plate at room temperature for 5 minutes. The assay plate was read using BMG PheraStar (Ex. 340 nm, En. 615 nm). Results are shown in FIG. 5.

### Example 4. Determination of the Melting Temperature of TSHR Antibodies

A factor in selecting a variant TSHR antibody was melting temperature. A desired variant antibody should have a similar, if not higher, melting temperature. The melting temperature of the 5 variants was measured with differential scanning fluorimetry (DSF) and compare to the parental antibody. The results of the second melting temperature (Tm2) are reported below in Table 2.

TABLE 2
Melting Temperatures

| Antibody | DSF Tm2 (° C.) |
|---|---|
| Parental | 70.5 |
| ETY-77 | 74.5 |
| ETY-86 | 73.5 |
| ETY-94 | 82 |
| ETY-98 | 79 |
| ETY-155 | 72 |

As shown in Table 2, each of the 5 variants possessed an elevated melting temperature relative to the parental antibody, with ETY-94 and ETY-98 exceeding the parental melting temperature by over 8° C.

### Example 5. Determining In Vitro Activity of TSHR Antibodies Against TSHR Signaling

The antibodies were screened for blocking of agonist activity using a TSHR activity reporter cell line. Intracellular cAMP levels were measured as an indicator of the ability of the antibodies to block autoimmune antibodies or TSH from TSHR-induced signaling. cAMP levels were also used as a measurement of agonistic activity and blocking of an agonist TSHR antibody or Graves' disease patient sera or antibodies purified from that sera-induced production of cAMP. The assay was performed with the variant antibodies co-incubated with the thyroid stimulating autoantibody M22, which mimics TSH binding to TSHR (see, Miguel et al. J Mol Endocrinol. 2009. 42(5):381-95). HiTSeeker HEK293-TSHR cell line (Innoprot #P30199) cell line was grown in DMEM medium supplemented with 10% FCS, 1% Pen-Strep and 0.25 mg/mL G418 (Geneticin, Gibco #10131-035) at 37° C., 5% CO2. 6000 cells were seeded into 384-well LV plate (Greiner #3284). Cells were incubated with increasing concentrations, from 0-200 nM, of candidate antibodies in HBSS alone for 15 min at 37° C. and then stimulated with 0.2 nM M22 for 45 min at 37° C. The cells and cAMP standard dilutions were further processed using HTRF cAMP kit (Cisbio #62AM4PEB) according to the manufacturer's instructions.

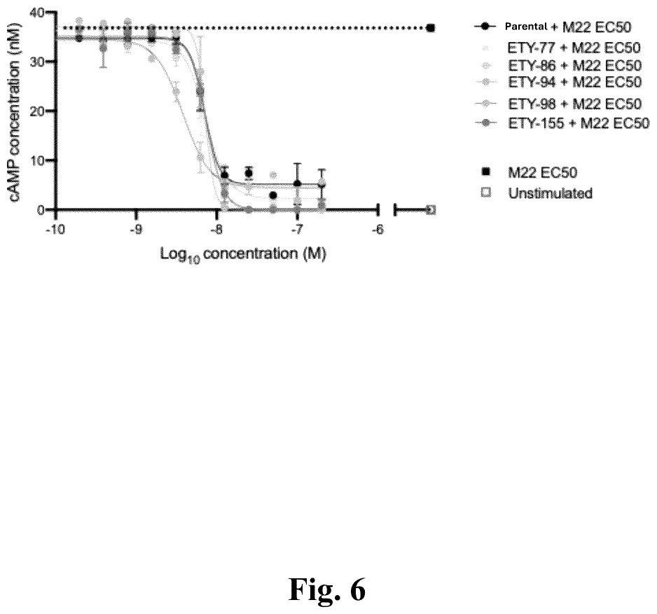

The results of the assay are depicted in FIG. 6, which demonstrates that the variant antibodies retain the activity of blocking agonist-induced cAMP production compared to the parental antibody, while greatly reducing the undesirable CD80 off-target binding.

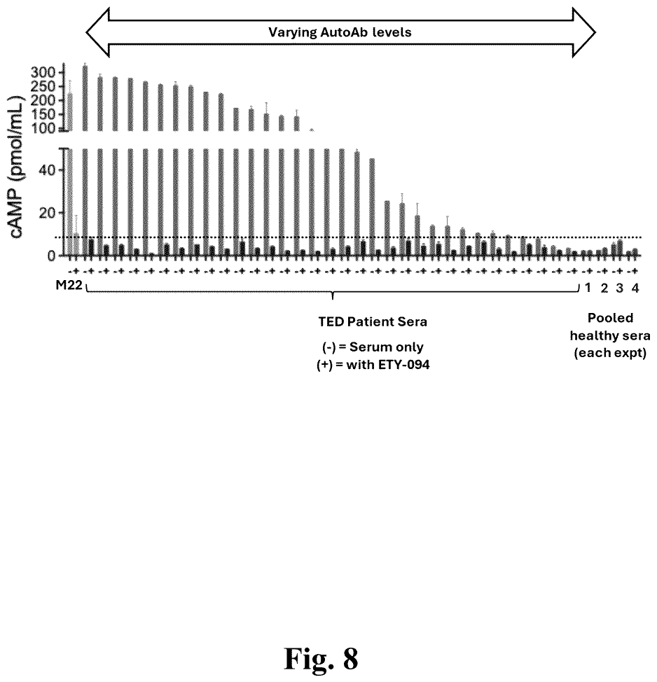

A similar assay cAMP assay was performed using 34 Moderate-to-Severe TED patients polyclonal sera samples, each with varying levels of TSHR auto antibodies. The assay tested the TSHR antibody ETY-94. As shown in FIG. 8, ETY-94 led to complete blocking of cAMP production in every TED patient sample tested.

### Human TED Orbital Fibroblast Assay

An assay involving orbital fibroblasts was employed as a way to interrogate the activity of the antibodies of the present disclosure.

### Materials and Methods

### Human TED Orbital Fibroblast Culture

Primary human orbital fibroblasts were established from thyroid eye disease (TED) orbital tissue obtained from surgical decompression surgery under institutional approval from the University of Rochester Medical Center and in accordance with the Declaration of Helsinki. Cells are maintained in fibroblast growth medium supplemented with 10% fetal bovine serum and antibiotics. For adipocyte differentiation, cells are cultured in adipogenic induction medium containing insulin, dexamethasone, IBMX, and rosiglitazone for 21 days. Differentiation is confirmed by morphological changes and increased TSHR expression measured by qPCR.

### Treatments

Differentiated TED adipocytes were treated with the stimulatory anti-TSHR monoclonal antibody M22 (10-30 nM), alone or in combination with pharmacologic inhibitors (Teprotumumab, K1-70, or ETY-94) as indicated for each experimental condition. Vehicle controls were included in all experiments. Following treatment initiation, cultures were maintained for 4 days. Conditioned media were collected at the end of the 4 days, clarified by centrifugation to remove debris, and stored at −80° C. until analysis.

### Hyaluronic Acid (HA) Detection

HA production is measured using a commercially available ELISA kit (Echelon Biosciences, Salt Lake City, UT), based on the specific interaction between HA and immobilized hyaluronan-binding protein (HABP). Cell culture supernatants are diluted and incubated in HABP-coated microplates, followed by sequential incubation with biotinylated HABP and streptavidin-conjugated HRP. Colorimetric detection is performed using a chromogenic substrate, and absorbance is measured at 450 nm on a Varioskan Flash plate reader (Thermo Fisher Scientific). To confirm assay specificity, selected samples are pretreated with hyaluronidase (2 U/mL, 2 h, 37° C.) prior to analysis.

### IL-6 Production

IL-6 levels are assessed using sandwich ELISA. Conditioned media are collected after treatment, centrifuged to remove debris, and applied to ELISA plates coated with capture antibodies against IL-6 (BD Pharmingen). After blocking, diluted media are incubated for 2 hours at room temperature, followed by the addition of biotinylated detection antibodies (IL-6: BD Pharmingen) and streptavidin-conjugated AP (Bio-Rad). PNPP substrate is added, and absorbance is measured at 405 nm with a reference wavelength of 690 nm using an iMark microplate reader (Bio-Rad). Cytokine concentrations are interpolated from standard curves generated using recombinant protein standards. All samples are measured in technical triplicate and normalized to cell number or total protein.

### Data Processing, Normalization, and Statistical Analysis

ELISA concentrations were compiled and analyzed in GraphPad Prism. Data were graphed as individual biological replicates with summary statistics as indicated (mean±SEM unless otherwise stated). To enable comparison across independent experiments, values were linearly rescaled within each experiment such that the appropriate control condition (vehicle or untreated) was set to 20% and the maximal response condition within that experiment was set to 100%; all other conditions were scaled proportionally. Statistical testing was performed in Prism using one-way ANOVA followed by Dunnett's multiple-comparisons test, with α=0.05.

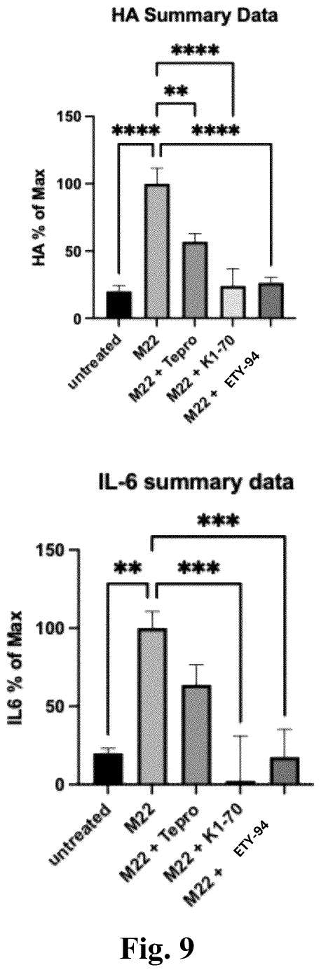

As shown in FIG. 9, ETY-94 reduced HA and IL-6 levels to a substantially greater degree than the current approved therapy for TED, teprotumumab.

### Example 6. T Cell Epitope Screening

The presence of putative HLA class II restricted epitopes, also known as Tₕ epitopes, of 6 antibodies was compared. Specifically, the sequences of ETY-cont (the parental TSHR antibody having a VH domain of SEQ ID NO: 61 and a VL domain of SEQ ID NO: 62), ETY-77, ETY-86, ETY-94, ETY-98 and ETY-155 were subjected to immunoprofiling using the Epibase® platform's “HLA class II—Global v4.0” settings.

Profiling was performed at the allotype level for 43 HLA-DRB1, 8 HLA-DRB3/4/5, 22 HLA-DQ and 12 HLA-DP, i.e., 85 HLA class II receptors in total. Table 3 lists the predicted number of critical DRB1, DRB3/4/5, DQ and DP epitopes for each molecule. Peptides binding to multiple allotypes of the same group are only counted once.

TABLE 3
Critical epitope counts per gene family. Numbers
between brackets refer to self-epitopes.

| Antibody | DRB1 | DRB3/4/5 | DQ | DP |
|---|---|---|---|---|
| ETY-cont | 34 (142) | 22 (72) | 1 (8) | 0 (6) |
| ETY-77 | 29 (151) | 20 (74) | 2 (8) | 0 (7) |
| ETY-86 | 29 (151) | 20 (74) | 2 (8) | 0 (7) |
| ETY-94 | 31 (150) | 21 (73) | 1 (10) | 0 (7) |
| ETY-98 | 29 (152) | 19 (74) | 2 (10) | 0 (7) |
| ETY-155 | 25 (149) | 15 (73) | 1 (10) | 0 (7) |

The allotypes affected by critical epitopes identified in the analyzed antibodies are shown in Table 4, together with their serotype and Global population frequencies.

TABLE 4
Affected HLA allotypes considered in the epitope profiling. For each HLA allotype,
its corresponding serotype and frequency in the Global population are shown,
as well as the total critical epitope count for each of the 6 antibodies.

| Allotype | Serotype | Freq (%) | ETY-cont | ETY-77 | ETY-86 | ETY-94 | ETY-98 | ETY-155 |
|---|---|---|---|---|---|---|---|---|
| DRB1*01:01 | DR1 | 6.6% | 6 | 4 | 5 | 4 | 4 | 1 |
| DRB1*01:02 | DR1 | 2.4% | 7 | 8 | 8 | 4 | 5 | 3 |
| DRB1*03:01 | DR17(3) | 13.0% | 3 | 4 | 4 | 5 | 3 | 2 |
| DRB1*03:02 | DR17(3) | 1.5% | 3 | 4 | 4 | 4 | 3 | 3 |
| DRB1*03:04 | DR17(3) | 1.0% | 3 | 4 | 4 | 5 | 3 | 2 |
| DRB1*04:01 | DR4 | 4.8% | 4 | 3 | 3 | 2 | 2 | 2 |
| DRB1*04:03 | DR4 | 3.2% | 3 | 2 | 2 | 3 | 2 | 4 |
| DRB1*04:04 | DR4 | 2.9% | 3 | 2 | 2 | 3 | 2 | 4 |
| DRB1*04:05 | DR4 | 5.5% | 4 | 4 | 4 | 4 | 4 | 4 |
| DRB1*04:06 | DR4 | 1.8% | 3 | 3 | 3 | 4 | 3 | 4 |
| DRB1*04:07 | DR4 | 1.3% | 5 | 3 | 3 | 3 | 3 | 4 |
| DRB1*04:10 | DR4 | 0.6% | 3 | 4 | 4 | 5 | 5 | 5 |
| DRB1*04:11 | DR4 | 0.3% | 3 | 4 | 4 | 5 | 5 | 5 |
| DRB1*07:01 | DR7 | 16.4% | 5 | 6 | 6 | 5 | 5 | 3 |
| DRB1*08:01 | DR8 | 1.7% | 4 | 3 | 3 | 4 | 4 | 3 |
| DRB1*08:02 | DR8 | 2.0% | 7 | 4 | 4 | 3 | 3 | 2 |
| DRB1*08:03 | DR8 | 5.9% | 4 | 3 | 3 | 4 | 4 | 3 |
| DRB1*08:04 | DR8 | 1.5% | 7 | 4 | 4 | 3 | 3 | 2 |
| DRB1*09:01 | DR9 | 10.8% | 5 | 3 | 4 | 1 | 2 | 0 |
| DRB1*10:01 | DR10 | 4.6% | 4 | 4 | 4 | 2 | 2 | 1 |
| DRB1*11:01 | DR11(5) | 10.5% | 2 | 2 | 2 | 2 | 2 | 2 |
| DRB1*11:02 | DR11(5) | 1.5% | 6 | 4 | 4 | 3 | 3 | 4 |
| DRB1*11:04 | DR11(5) | 2.4% | 1 | 1 | 1 | 1 | 1 | 1 |
| DRB1*11:06 | DR11(5) | 0.9% | 1 | 1 | 1 | 1 | 1 | 1 |
| DRB1*11:10 | DR11(5) | 0.3% | 2 | 2 | 2 | 2 | 2 | 2 |
| DRB1*12:01/12:06/12:10 | DR12(5) | 4.5% | 3 | 3 | 3 | 2 | 3 | 3 |
| DRB1*12:02 | DR12(5) | 10.8% | 3 | 3 | 3 | 2 | 3 | 3 |
| DRB1*13:01 | DR13(6) | 6.4% | 5 | 5 | 6 | 3 | 4 | 5 |
| DRB1*13:02 | DR13(6) | 6.7% | 4 | 4 | 5 | 3 | 4 | 5 |
| DRB1*13:03 | DR13(6) | 2.8% | 2 | 3 | 3 | 2 | 2 | 2 |
| DRB1*13:04 | DR13(6) | 0.4% | 3 | 2 | 2 | 3 | 3 | 5 |
| DRB1*13:12 | DR13(6) | 0.8% | 2 | 3 | 3 | 2 | 2 | 2 |
| DRB1*14:01/14:54 | DR14(6) | 6.9% | 3 | 3 | 3 | 2 | 2 | 2 |
| DRB1*14:02 | DR14(6) | 0.7% | 3 | 3 | 3 | 1 | 1 | 1 |
| DRB1*14:04 | DR14(6) | 2.0% | 3 | 3 | 3 | 2 | 2 | 2 |
| DRB1*14:05 | DR14(6) | 1.6% | 1 | 1 | 1 | 1 | 1 | 1 |
| DRB1*14:06 | DR14(6) | 0.6% | 5 | 4 | 5 | 3 | 3 | 2 |
| DRB1*15:01 | DR15(2) | 14.0% | 8 | 8 | 8 | 6 | 7 | 5 |
| DRB1*15:02 | DR15(2) | 8.8% | 5 | 5 | 6 | 6 | 6 | 5 |
| DRB1*15:03 | DR15(2) | 3.7% | 8 | 8 | 8 | 6 | 7 | 5 |
| DRB1*15:04 | DR15(2) | 1.2% | 8 | 8 | 8 | 6 | 7 | 5 |
| DRB1*16:01 | DR16(2) | 1.4% | 6 | 7 | 7 | 6 | 7 | 6 |
| DRB1*16:02 | DR16(2) | 4.0% | 6 | 7 | 7 | 6 | 7 | 6 |
| DRB3*01:01 | DR52 | 10.9% | 7 | 6 | 5 | 6 | 4 | 5 |
| DRB3*02:01 | DR52 | 2.1% | 10 | 10 | 9 | 8 | 8 | 7 |
| DRB3*02:02 | DR52 | 23.3% | 8 | 6 | 6 | 5 | 5 | 5 |
| DRB3*03:01 | DR52 | 10.4% | 10 | 11 | 11 | 9 | 10 | 9 |
| DRB4*01:01/01:03/01:06 | DR53 | 27.8% | 3 | 3 | 3 | 2 | 2 | 2 |
| DRB5*01:01 | DR51 | 12.1% | 5 | 4 | 4 | 5 | 4 | 3 |
| DRB5*01:02 | DR51 | 5.5% | 2 | 1 | 2 | 2 | 3 | 2 |
| DRB5*02:02 | DR51 | 1.4% | 6 | 4 | 6 | 4 | 5 | 2 |
| DQA1*01:02\|DQB1*06:04 | DQ6(1) | 1.6% | 0 | 1 | 1 | 0 | 1 | 0 |
| DQA1*01:02\|DQB1*06:09 | DQ6(1) | 1.1% | 0 | 1 | 1 | 0 | 1 | 0 |
| DQA1*01:03\|DQB1*06:09 | DQ6(1) | 0.4% | 0 | 1 | 1 | 0 | 1 | 0 |
| DQA1*03:01/03:02/03:03\| | DQ7(3) | 1.7% | 1 | 1 | 1 | 1 | 1 | 0 |
| DQB1*03:01/03:09/03:19 | | | | | | | | |
| DQA1*04:01\|DQB1*03:01/ | DQ7(3) | 0.6% | 1 | 1 | 1 | 1 | 1 | 1 |
| 03:09/03:19 | | | | | | | | |
| DQA1*05:01 g\|DQB1*03:01/ | DQ7(3) | 13.0% | 1 | 1 | 1 | 1 | 1 | 1 |
| 03:09/03:19 | | | | | | | | |
| DQA1*06:01/06:02\|DQB1 | DQ7(3) | 0.9% | 1 | 1 | 1 | 1 | 1 | 1 |
| *03:01/03:09/03:19 | | | | | | | | |

| | | | | | | |
|---|---|---|---|---|---|---|
| Total number of affected DRB1 allotypes | 43 | 43 | 43 | 43 | 43 | 42 |
| Total number of affected DRB3/4/5 allotypes | 8 | 8 | 8 | 8 | 8 | 8 |
| Total number of affected DQ allotypes | 4 | 7 | 7 | 4 | 7 | 3 |
| Total number of affected DP allotypes | 0 | 0 | 0 | 0 | 0 | 0 |

An approximate score expressing a worst-case immunogenic risk can be calculated as follows:

$$DRB 1 score = \sum ( epitope count \times allotype frequency )$$

The number of critical epitopes affecting a particular HLA allotype is multiplied by the allele frequency of the affected allotype. For a given sequence, the products are summed for all DRB1 allotypes used in the study. Table 5 represents the DRB1 risk scores in the Global population for the antibodies analyzed in this study.

TABLE 5
DRB1 risk scores of the 6 antibodies in the Global population.

Global DRB1 risk score

| Antibodies | Heavy chain | Light chain | Full antibodies |
|---|---|---|---|
| ETY-cont | 610.7 | 183.6 | 794.3 |
| ETY-77 | 557.3 | 210.8 | 768.1 |
| ETY-86 | 557.3 | 250.7 | 808.0 |
| ETY-94 | 431.5 | 217.1 | 648.6 |
| ETY-98 | 408.1 | 269.0 | 677.1 |
| ETY-155 | 408.1 | 138.4 | 546.5 |

### Conclusions:

### Critical Epitope Counts:

The data from Table 3 shows that ETY-155 has the lowest number of critical DRB1 epitopes (25 epitopes) and ETY-cont has the highest (34 epitopes). Similarly, for the DRB3/4/5 family, ETY-155 has the lowest epitope count (15 epitopes) and ETY-cont has the highest with 22 epitopes. Three antibodies, ETY-cont, ETY-94 and ETY-155 have one DQ epitope, and ETY-77, ETY-86 and ETY-98 have the highest number of epitopes (2 epitopes). For the DP family, none of the 6 antibodies have any DP epitopes.

### Affected Allotypes:

For the DRB1 family, ETY-155 affect the lowest number of allotypes, 42 out of 43. Other 5 antibodies affect all the DRB1 allotypes, 43 out of 43. Table 4 shows that for the majority of affected DRB1 allotypes, ETY-155 has the lowest number of critical epitopes. Allotypes of serotypes DR15(2) and DR16(2) are most affected by ETY-86, including the prevalent allotypes DRB1*15:01 and DRB1*15:02, which are present in 14.0% and 8.8% of the Global population, respectively. ETY-155 affects DR4 allotypes with higher epitope counts. For the DRB3/4/5 family, all 6 antibodies affect 8 (out of 8) allotypes. ETY-155 has the lowest epitope counts for the DRB3/4/5 allotypes. ETY-155 affects 3 DQ allotypes. ETY-cont and ETY-94 affect 4 DQ allotypes each. The other 3 antibodies affect the highest number of DQ allotypes (7 allotypes out of 22). For each affected allotype the epitope counts are low. For the DP family, all 6 antibodies do not affect any allotypes.

### DRB1 Score:

The data in Table 5 shows that ETY-155 has the lowest DRB1 score. Next, in the order of increasing score, are ETY-94 and ETY-98 with similar scores; the three antibodies (ETY-cont, ETY-77, ETY-86) have at least a 40% increase in DRB1 scores when compared to the ETY-77 score.

The main contributors to the score in all 6 antibodies are the CDRs H1 and H3. The substitution(s) made in the CDR L1 is a major contributor in 4 out of 5 antibodies (ETY-77, ETY-86, ETY-94 and ETY-98). The substitutions made in the CDR L2 chain of ETY-155 is also a major contributor. The Fc substitutions M252Y/S254T/T256E (EU numbering) in the 5 antibodies contribute moderately to the score. Other substitutions in the 5 antibodies with respect to ETY-cont either decrease the score or do not significantly contribute to the score.

### Ranking by Predicted Immunogenicity:

Based on the critical epitope counts, the affected HLA allotypes and DRB1 risk score, the rank of the 6 antibodies by increased immunogenic risk as follows:

$$ETY - 155 < ETY - 94 < ETY - 98 < ETY - 77 < [ ETY - cont , ETY - 86 ]$$

### Example 7. Off-Target Binding of TSHR Antibodies from a Membrane Proteome Array (MPA)

The Membrane Proteome Array (MPA) is a platform for profiling the specificity of antibodies and other ligands that target human membrane proteins. The MPA can be used to determine antibody target specificity and deconvolute orphan antibody/ligand targets. The platform uses flow cytometry to directly detect antibody binding to membrane proteins expressed in unfixed cells. Consequently, all target proteins have native conformations and appropriate post-translational modifications. The TSHR antibodies ETY-77, ETY-86, ETY-94, ETY-98, ETY-155, and the parental antibody were tested for reactivity against a library of over 6,000 human membrane proteins, including 94% of all single-pass, multi-pass, and GPI-anchored proteins, including GPCRs, ion channels, and transporters. Identified targets were validated in secondary screens to confirm reactivity.

To identify ligand binding targets, ~6,000 different membrane proteins were each expressed in individual wells of HEK-293T cells arrayed in 384-well plates. The cells were then matrixed by pooling individual columns and rows of each plate, so that each protein was represented in a unique combination of two different wells of the matrix plate. ETY-77, ETY-86, ETY-98, ETY-155, and the parental antibody were tested at 20 μg/mL while ETY-94 was tested at 5 μg/mL.

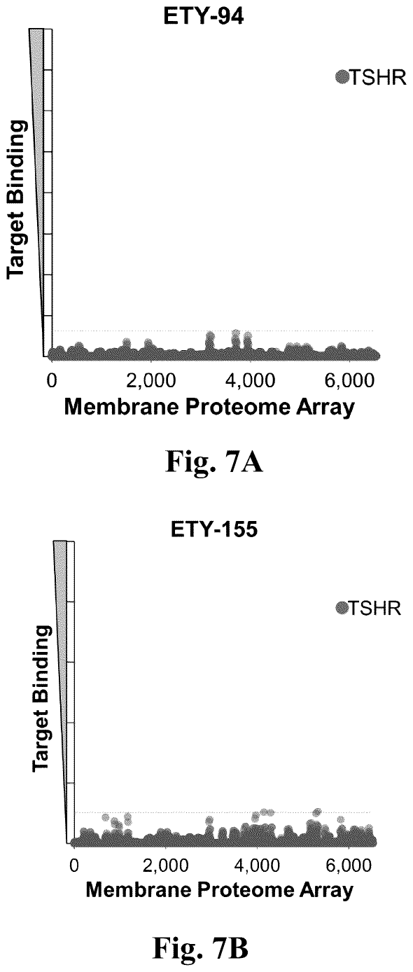

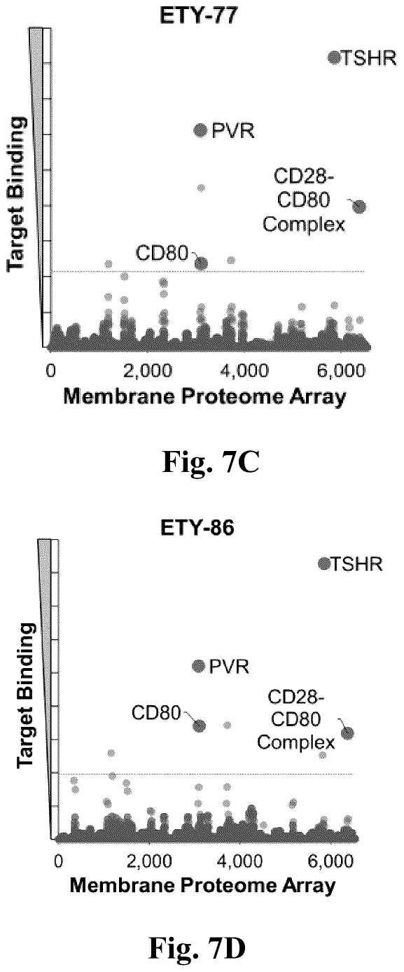

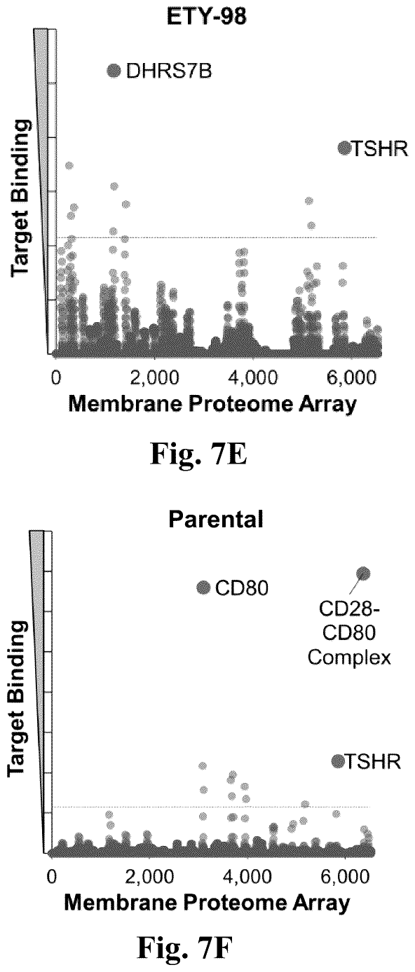

Targets were identified by detecting ligand binding to overlapping column and row pools, thereby allowing specific deconvolution. Each individual membrane protein target was assigned values corresponding to the binding values of their unique row and column pools, and targets demonstrating binding signal>3 standard deviations above background in both wells were selected for downstream validation experiments. The resulting paired binding values were subsequently normalized and transformed to give a single numerical value for binding of the test ligand against each target protein. Non-specific fluorescence was determined to be any value below 3 standard deviations of the mean background value. The results are shown in FIG. 7A-7F, which demonstrate that each of ETY-77, ETY-86, ETY-94, ETY-98, and ETY-155 had significantly reduced binding to CD80 compared to the parental antibody.

### Example 8. Characterization of a pH-Sensitive TSHR Antibody

In the development and screening of TSHR antibody variants described above, an antibody was identified that possessed differential binding to TSHR at alternative pH values. The antibody, designated ETY-67, exhibited a 26-fold loss in affinity and a 23-fold faster off-rate at pH 5.8 compared to pH 7.4. In contrast, the parental antibody only exhibited a 1.4-fold loss in affinity and a 2-fold faster off-rate at pH 5.8 compared to pH 7.4. Characterization is described further below.

Binding kinetics were measured by SPR using the Bruker MASS-2 system. A high-capacity amine (HCA) sensor (Bruker #1862614) immobilized with ≥10,000 RU anti-HuFc IgG1 (MP Bio #0855071) on spots AB was used. 10 nM of purified ETY-67 was injected onto spot B by injection for 45 sec at 25 μL/min, aiming for a capture level of 400 RU. Biotinylated TSHR22-260 WT, Avi His tag expressed at 27° C. was injected over spots AB for 4 min at 50 μL/min, followed by 20 min dissociation phase. Analyte concentrations of 12.5 nM, 25 nM, 50 nM, 100 nM, and 200 nM were injected in MCK-assay format, from lowest to highest. Regeneration was performed with 10 mM Glycine-HCl, pH 1.7 injected for 15 seconds at 25 μL/min. Buffer blank injections and reference spot (A) analyte injections were each subtracted to produce double-subtracted sensorgrams. 10 nM of large-scale purification of the parental antibody was used as a control for each SPR experiment. The pH 7.4 running buffer was 20 mM HEPES, 150 mM NaCl, 1.3 mM CaCl₂ with 0.05% v/v Tween® 20. The pH 5.8 running buffer was 20 mM HEPES, 150 mM NaCl, 0.1 mM CaCl₂ with 0.05% v/v Tween® 20. Data was analyzed using Bruker Analyzer 4 Software.

Binding kinetic data is reported below:

TABLE 6
Binding kinetic data

| | | Capture | | | |
|---|---|---|---|---|---|
| Antibody | Buffer | Level (RU) | K_{D} (M) | kₒₙ (M⁻¹s⁻¹) | k_{off} (s⁻¹) |
| Parental | PBS, pH 7.4 | 338.5 ± 101.4 | (5.84 ± | (9.25 ± | (5.32 ± |
| | | | 0.90) × 10⁻¹⁰ | 1.07) × 10⁴ | 0.27) × 10⁻⁵ |
| | HEPES, pH 7.4 | 324.7 ± 0.5 | 5.73 × 10⁻¹⁰ | 9.67 × 10⁴ | 5.54 × 10⁻⁵ |
| | HEPES, pH 5.8 | 573.0 ± 11.0 | 8.06 × 10⁻¹⁰ | 1.39 × 10⁵ | 1.12 × 10⁻⁴ |
| ETY-67 | PBS, pH 7.4 | 453.5 ± 8.7 | 1.35 × 10⁻⁹ | 8.88 × 10⁴ | 1.20 × 10⁻⁴ |
| | HEPES, pH 7.4 | 243.7 ± 4.9 | 1.35 × 10⁻⁹ | 8.33 × 10⁴ | 1.12 × 10⁻⁴ |
| | HEPES, pH 5.8 | 318.9 ± 5.7 | 4.14 × 10⁻⁸ | 6.45 × 10⁴ | 2.67 × 10⁻³ |

| Antibody | Buffer | t_{1/2} (s) | t_{1/2} (hh:mm:ss) | Rₘₐₓ (RU) | X²/Rₘₐₓ (%) |
|---|---|---|---|---|---|
| Parental | PBS, pH 7.4 | 13,063 ± 662 | 03:37:43 ± 00:11:02 | 132.3 ± 35.2 | 1.0 ± 0.4 |
| | HEPES, pH 7.4 | 12,502 | 03:28:22 | 143.0 | 5.5 |
| | HEPES, pH 5.8 | 6,182 | 01:43:02 | 262.1 | 16.7 |
| ETY-67 | PBS, pH 7.4 | 5,768 | 01:36:08 | 134.7 | 1.0 |
| | HEPES, pH 7.4 | 6,162 | 01:42:42 | 99.6 | 6.1 |
| | HEPES, pH 5.8 | 259 | 00:04:19 | 123.1 | 9.1 |

### Example 9. Measuring Various Characteristics of TSHR Antibodies for Developability

Graves' disease is an autoimmune disorder that presents with the production of autoantibodies that can bind to TSHR, thereby activating the receptor and causing hyperthyroidism. The antibodies of the present disclosure may block the autoantibodies produced by the subject without activating the receptor. Therefore, the extent to which the TSHR antibodies of the present disclosure block binding of isolated TSHR autoantibodies or autoantibodies in the sera of Graves' disease patients will be assessed.

The blocking of an autoimmune TSHR antibody by the antibodies of the present disclosure can be measured using flow cytometry and/or SPR/BLI or ELISA. Furthermore, patient sera from Graves' disease patients comprising autoimmune antibodies may be used to further measure the ability of the antibodies of the present disclosure to block the autoantibodies binding to TSHR-overexpressing cells. This can be measured with methods including flow cytometry and/or ELISA.

The anti-TSHR antibodies of the present disclosure will be further characterized for traits important to the developability of the antibodies for therapeutic use. The melting temperatures, serum stability, and stability at a low pH of the antibodies will be measured.

The antibodies' solubility and propensity for aggregation will also be tested. This can be tested employing hydrophobic interaction chromatography, dynamic light scattering (DLS), size-exclusion chromatography (SEC), or self-interaction (AC-SINS). The viscosity of the antibody in solution at a range of concentrations will also be measured.

Specificity of the antibodies will be tested by measuring binding to targets other than TSHR. Binding other proteins such as insulin, DNA baculovirus particles, or a cell lysate can be an indicator of the degree of polyreactivity of antibodies, and thus will be tested for the TSHR antibodies. The binding will be measured via ELISA, flow cytometry, BLI, or other methods as a predictor for off-target binding that could also lead to poor PK. Undesired binding to specific off-target proteins can be measured, as detected via a screen of binding to fixed cells expressing a single membrane or tethered secreted protein from a human protein library using a library from Retrogenix (Charles River Laboratories) or other similar technology. This assay can be followed up with measuring the binding to the off-target protein by a number of different assays such as SPR, BLI, ELISA, and/or flow cytometry.

Antibody degradation due to temperature, freeze-thaw cycles, agitation, pH, and other common conditions will be measured. The antibody sequences will be interrogated for liabilities where amino acid substitutions may improve any of the above conditions or characteristics.

### Example 10. Testing the Immunogenicity of TSHR Antibodies

An effort to reduce the immunogenicity of the TSHR antibodies may be made. Anti-drug antibodies (ADAs) may affect the risk profile and efficacy of a biological drug. Therefore, the level of ADAs in normal human serum that may bind to the TSHR antibodies of the present disclosure may be tested using ELISA or similar methodology. Additionally, the sera of Graves' disease patients may also be tested for ADA in the same manner. Computational predictions of T cell epitopes and/or primary human T cell or peripheral blood mononuclear cell (PBMC) assays may also be used to assess the predicted immunogenicity of the TSHR antibodies.

Additional anti-drug antibody assays are e.g., detailed in WO2007101661A1 (Hoffmann La Roche), WO2018178307A1 (Ablynx), WO2021046316A2 (Adverum Biotechnologies, Charles River), and US20180088140A1 (Genzyme Corporation), each of which is incorporated herein by reference.

### Example 11. Determining the Pharmacokinetics/Pharmacodynamics (PK/PD) in an In Vivo Model

Variable region (Fv) amino acid sequences in either CDRs and/or framework regions may affect cellular uptake and recycling (PK) of the anti-TSHR antibodies due to faster non-specific clearance due to undesired electrostatic interactions between the charged Fv and negatively charged components of vascular and/or tissue cells or by modulation of FcRn binding affinity and subsequent recycling of the antibody. Assessment of potential PK differences in a relevant pre-clinical animal model is desired prior to therapeutic administration in human subjects.

Potential differences in PK parameters (e.g., AUC, half-life, Cmax, Tmax, CI, and F) may be measured in a mouse, rat, and/or non-human primate (NHP) models. In mice, a transgenic FcRn mouse (Tg32) may be used in addition. Different dose levels (e.g. high, med, and low) will be administered using various routes (IV, SC) that will generate a range of appropriate exposures that are expected to be achieved in humans. The concentration of anti-TSHR antibody in serum will be measured using ELISA on blood samples taken at various timepoints before and after dosing. An appropriate anti-human IgG ELISA and standard curve will be employed to detect and quantitate human IgG in mouse, rat, or NHP sera. Pharmacokinetic analyses (noncompartmental) will be performed using appropriate tools such as Phoenix WinNonlin software or equivalent.

Potential differences in pharmacodynamic activity (e.g. receptor occupancy, potency, efficacy) may also be measured in rat and NHP models.

Measurements to observe T3, T4, and elevated TSH serum levels may be made to detect and quantitate the ability of anti-TSHR antibodies to inhibit TSH binding and subsequent signaling through the TSHR thus inhibiting thyroid function (biochemical hypothyroidism) in these preclinical models. Different dose levels (e.g. high, med, and low) will be administered using various routes (IV, SC) that will generate a range of appropriate exposures that are expected to be achieved in humans. The concentration of thyroid hormones (T3, T4, TSH) in serum will be measured in blood samples taken at various timepoints before and after dosing. An appropriate anti-rat or anti-NHP thyroid hormone ELISA and standard curve will be employed to detect and quantitate levels in rat or NHP sera. Other immunoassays formats may also be employed depending on species and sensitivity considerations.

SEQUENCES

| | SEQ | |
|---|---|---|
| Antibody | ID NO | Sequence |
| ETY-77 | 1 | GYSLTDNW |
| HCDR1 | | |
| ETY-77 | 2 | IYPGDSDT |
| HCDR2 | | |
| ETY-77 | 3 | VGLDWNYNPLRY |
| HCDR3 | | |
| ETY-77 | 4 | LSDIGSNY |
| LCDR1 | | |
| ETY-77 | 5 | DNNKRPS |
| LCDR2 | | |
| ETY-77 | 6 | GTWDSRLGIAV |
| LCDR3 | | |
| ETY-77 VH | 7 | EVQLVQSGAEVKKPGESLKISCKASGYSLTDNWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSS |
| ETY-77 VL | 8 | QSVLTQPPSVSAAPGQKVTISCSGSLSDIGSNYVSWYQQLP |
| | | GTAPKLLIYDNNKRPSGIPDRFSGSKSGTSATLGITGLQTGD |
| | | EADYYCGTWDSRLGIAVFGGGTQLTVL |
| ETY-77 HC | 9 | EVQLVQSGAEVKKPGESLKISCKASGYSLTDNWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPELLGGPSVFLFP |
| | | PKPKDTLYITREPEVTCVVVDVSHEDPEVKFNWYVDGVEV |
| | | HNAKTKPREEQYNSTYRVVSVLTVLHQDWLNGKEYKCKV |
| | | SNKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| | | K |
| ETY-77 LC | 10 | QSVLTQPPSVSAAPGQKVTISCSGSLSDIGSNYVSWYQQLP |
| | | GTAPKLLIYDNNKRPSGIPDRFSGSKSGTSATLGITGLQTGD |
| | | EADYYCGTWDSRLGIAVFGGGTQLTVLGQPKAAPSVTLFPP |
| | | SSEELQANKATLVCLVSDFYPGAVTVAWKADGSPVKVGVE |
| | | TTKPSKQSNNKYAASSYLSLTPEQWKSHRSYSCRVTHEGST |
| | | VEKTVAPTECS |
| ETY-86 | 11 | GYSLTDNW |
| HCDR1 | | |
| ETY-86 | 12 | IYPGDSDT |
| HCDR2 | | |
| ETY-86 | 13 | VGLDWNYNPLRY |
| HCDR3 | | |
| ETY-86 | 14 | LRDIGSNY |
| LCDR1 | | |
| ETY-86 | 15 | DNNKRPS |
| LCDR2 | | |
| ETY-86 | 16 | GTWDSRLGIAV |
| LCDR3 | | |
| ETY-86 VH | 17 | EVQLVQSGAEVKKPGESLKISCKASGYSLTDNWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSS |
| ETY-86 VL | 18 | QSVLTQPPSVSAAPGQKVTISCSGSLRDIGSNYVSWYQQLP |
| | | GTAPKLLIYDNNKRPSGIPDRFSGSKSGTSATLGITGLQTGD |
| | | EADYYCGTWDSRLGIAVFGGGTQLTVL |
| ETY-86 HC | 19 | EVQLVQSGAEVKKPGESLKISCKASGYSLTDNWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPELLGGPSVFLFP |
| | | PKPKDTLYITREPEVTCVVVDVSHEDPEVKFNWYVDGVEV |
| | | HNAKTKPREEQYNSTYRVVSVLTVLHQDWLNGKEYKCKV |
| | | SNKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| | | K |
| ETY-86 LC | 20 | QSVLTQPPSVSAAPGQKVTISCSGSLRDIGSNYVSWYQQLP |
| | | GTAPKLLIYDNNKRPSGIPDRFSGSKSGTSATLGITGLQTGD |
| | | EADYYCGTWDSRLGIAVFGGGTQLTVLGQPKAAPSVTLFPP |
| | | SSEELQANKATLVCLVSDFYPGAVTVAWKADGSPVKVGVE |
| | | TTKPSKQSNNKYAASSYLSLTPEQWKSHRSYSCRVTHEGST |
| | | VEKTVAPTECS |
| ETY-94 | 21 | GYSLTDNW |
| HCDR1 | | |
| ETY-94 | 22 | IYPGDSTT |
| HCDR2 | | |
| ETY-94 | 23 | VGLDWNYNPLRY |
| HCDR3 | | |
| ETY-94 | 24 | YSDIGSNY |
| LCDR1 | | |
| ETY-94 | 25 | DNNKRPS |
| LCDR2 | | |
| ETY-94 | 26 | GTWDSRLGIAV |
| LCDR3 | | |
| ETY-94 VH | 27 | EVQLVQSGAEVKKPGASVKVSCKASGYSLTDNWIGWVRQ |
| | | APGQGLEWMGIIYPGDSTTRYAQKFQGRVTMTADKSTSTV |
| | | YMELSSLRSEDTAVYYCVGLDWNYNPLRYWGQGTLVTVS |
| | | S |
| ETY-94 VL | 28 | QSVLTQPPSVSGAPGQRVTISCSGSYSDIGSNYVSWYQQLP |
| | | GTAPKLLIYDNNKRPSGVPDRFSGSKSGTSASLAITGLQAED |
| | | EADYYCGTWDSRLGIAVFGGGTKLTVL |
| ETY-94 HC | 29 | EVQLVQSGAEVKKPGASVKVSCKASGYSLTDNWIGWVRQ |
| | | APGQGLEWMGIIYPGDSTTRYAQKFQGRVTMTADKSTSTV |
| | | YMELSSLRSEDTAVYYCVGLDWNYNPLRYWGQGTLVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLYITREPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLS |
| | | LSPGK |
| ETY-94 LC | 30 | QSVLTQPPSVSGAPGQRVTISCSGSYSDIGSNYVSWYQQLP |
| | | GTAPKLLIYDNNKRPSGVPDRFSGSKSGTSASLAITGLQAED |
| | | EADYYCGTWDSRLGIAVFGGGTKLTVLGQPKAAPSVTLFPP |
| | | SSEELQANKATLVCLVSDFYPGAVTVAWKADGSPVKVGVE |
| | | TTKPSKQSNNKYAASSYLSLTPEQWKSHRSYSCRVTHEGST |
| | | VEKTVAPTECS |
| ETY-98 | 31 | GYSLTDNW |
| HCDR1 | | |
| ETY-98 | 32 | IYPGDSDT |
| HCDR2 | | |
| ETY-98 | 33 | VGLDWNYNPLRY |
| HCDR3 | | |
| ETY-98 | 34 | LRDIGSNY |
| LCDR1 | | |
| ETY-98 | 35 | DNNKRPS |
| LCDR2 | | |
| ETY-98 | 36 | GTWDSRLGIAV |
| LCDR3 | | |
| ETY-98 VH | 37 | EVQLVQSGAEVKKPGASVKVSCKASGYSLTDNWIGWVRQ |
| | | APGQGLEWMGIIYPGDSDTRYAQKFQGRVTMTADKSTSTV |
| | | YMELSSLRSEDTAVYYCVGLDWNYNPLRYWGQGTLVTVS |
| | | S |
| ETY-98 VL | 38 | QSVLTQPPSVSGAPGQRVTISCSGSLRDIGSNYVSWYQQLP |
| | | GTAPKLLIYDNNKRPSGVPDRFSGSKSGTSASLAITGLQAED |
| | | EADYYCGTWDSRLGIAVFGGGTKLTVL |
| ETY-98 HC | 39 | EVQLVQSGAEVKKPGASVKVSCKASGYSLTDNWIGWVRQ |
| | | APGQGLEWMGIIYPGDSDTRYAQKFQGRVTMTADKSTSTV |
| | | YMELSSLRSEDTAVYYCVGLDWNYNPLRYWGQGTLVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLYITREPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLS |
| | | LSPGK |
| ETY-98 LC | 40 | QSVLTQPPSVSGAPGQRVTISCSGSLRDIGSNYVSWYQQLP |
| | | GTAPKLLIYDNNKRPSGVPDRFSGSKSGTSASLAITGLQAED |
| | | EADYYCGTWDSRLGIAVFGGGTKLTVLGQPKAAPSVTLFPP |
| | | SSEELQANKATLVCLVSDFYPGAVTVAWKADGSPVKVGVE |
| | | TTKPSKQSNNKYAASSYLSLTPEQWKSHRSYSCRVTHEGST |
| | | VEKTVAPTECS |
| ETY-155 | 41 | GYSLTDNW |
| HCDR1 | | |
| ETY-155 | 42 | IYPGDSDT |
| HCDR2 | | |
| ETY-155 | 43 | VGLDWNYNPLRY |
| HCDR3 | | |
| ETY-155 | 44 | SSNVGSNY |
| LCDR1 | | |
| ETY-155 | 45 | DNNYVPS |
| LCDR2 | | |
| ETY-155 | 46 | GTWDSRLDAAV |
| LCDR3 | | |
| ETY-155 VH | 47 | EVQLVQSGAEVKKPGASVKVSCKASGYSLTDNWIGWVRQ |
| | | APGQGLEWMGIIYPGDSDTRYAQKFQGRVTMTADKSTSTV |
| | | YMELSSLRSEDTAVYYCVGLDWNYNPLRYWGQGTLVTVS |
| | | S |
| ETY-155 VL | 48 | QSVLTQPPSVSGAPGQRVTISCSGSSSNVGSNYVSWYQQLP |
| | | GTAPKLLIYDNNYVPSGVPDRFSGSKSGTSASLAITGLQAE |
| | | DEADYYCGTWDSRLDAAVFGGGTKLTVL |
| ETY-155 HC | 49 | EVQLVQSGAEVKKPGASVKVSCKASGYSLTDNWIGWVRQ |
| | | APGQGLEWMGIIYPGDSDTRYAQKFQGRVTMTADKSTSTV |
| | | YMELSSLRSEDTAVYYCVGLDWNYNPLRYWGQGTLVTVS |
| | | SASTKGPSVFPLAPSSKSTSGGTAALGCLVKDYFPEPVTVS |
| | | WNSGALTSGVHTFPAVLQSSGLYSLSSVVTVPSSSLGTQTYI |
| | | CNVNHKPSNTKVDKKVEPKSCDKTHTCPPCPAPELLGGPS |
| | | VFLFPPKPKDTLYITREPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTKN |
| | | QVSLTCLVKGFYPSDIAVEWESNGQPENNYKTTPPVLDSDG |
| | | SFFLYSKLTVDKSRWQQGNVFSCSVMHEALHNHYTQKSLS |
| | | LSPGK |
| ETY-155 LC | 50 | QSVLTQPPSVSGAPGQRVTISCSGSSSNVGSNYVSWYQQLP |
| | | GTAPKLLIYDNNYVPSGVPDRFSGSKSGTSASLAITGLQAE |
| | | DEADYYCGTWDSRLDAAVFGGGTKLTVLGQPKAAPSVTLF |
| | | PPSSEELQANKATLVCLVSDFYPGAVTVAWKADGSPVKVG |
| | | VETTKPSKQSNNKYAASSYLSLTPEQWKSHRSYSCRVTHEG |
| | | STVEKTVAPTECS |
| ETY-67 | 51 | GYHLTHHW |
| HCDR1 | | |
| ETY-67 | 52 | IYPGDSDT |
| HCDR2 | | |
| ETY-67 | 53 | VGLDWNYNPLRY |
| HCDR3 | | |
| ETY-67 | 54 | LSDIGSNY |
| LCDR1 | | |
| ETY-67 | 55 | DNNKRPS |
| LCDR2 | | |
| ETY-67 | 56 | GTWDSRLGIAV |
| LCDR3 | | |
| ETY-67 VH | 57 | EVQLVQSGAEVKKPGESLKISCKASGYHLTHHWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSS |
| ETY-67 VL | 58 | QSVLTQPPSVSAAPGQKVTISCSGSLSDIGSNYVSWYQQLP |
| | | GTAPKLLIYDNNKRPSGIPDRFSGSKSGTSATLGITGLQTGD |
| | | EADYYCGTWDSRLGIAVFGGGTQLTVL |
| ETY-67 HC | 59 | EVQLVQSGAEVKKPGESLKISCKASGYHLTHHWIGWVRQK |
| | | PGKGLEWMGIIYPGDSDTRYSPSFQGQVTISADKSISTAYLQ |
| | | WSSLKASDTAIYYCVGLDWNYNPLRYWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPELLGGPSVFLFP |
| | | PKPKDTLYITREPEVTCVVVDVSHEDPEVKFNWYVDGVEV |
| | | HNAKTKPREEQYNSTYRVVSVLTVLHQDWLNGKEYKCKV |
| | | SNKALPAPIEKTISKAKGQPREPQVYTLPPSRDELTKNQVSL |
| | | TCLVKGFYPSDIAVEWESNGQPENNYKTTPPVLDSDGSFFL |
| | | YSKLTVDKSRWQQGNVFSCSVMHEALHNHYTQKSLSLSPG |
| | | K |
| ETY-67 LC | 60 | QSVLTQPPSVSAAPGQKVTISCSGSLSDIGSNYVSWYQQLP |
| | | GTAPKLLIYDNNKRPSGIPDRFSGSKSGTSATLGITGLQTGD |
| | | EADYYCGTWDSRLGIAVFGGGTQLTVLGQPKAAPSVTLFPP |
| | | SSEELQANKATLVCLVSDFYPGAVTVAWKADGSPVKVGVE |
| | | TTKPSKQSNNKYAASSYLSLTPEQWKSHRSYSCRVTHEGST |
| | | VEKTVAPTECS |
| Parental VH | 61 | EVQLVQSGAEVKKPGQSLKISCKASGYSLTDNWIGWVRQK |
| | | PGKGLEWMGIIYPGDSDTRYSPSFQGQVTISADKSINTAYLQ |
| | | WSSLKASDTAIYYCVGLDWNYNPLRYWGPGTLVTVSS |
| Parental VL | 62 | QSVLTQPPSVSAAPGQKVTISCSGSSSDIGSNYVSWYQQFPG |
| | | TAPKLLIYDNNKRPSAIPDRFSGSKSGTSATLGITGLQTGDE |
| | | ADYYCGTWDSRLGIAVFGGGTQLTVL |
| Parental | 63 | GYSLTDNWIG |
| HCDR1 | | |
| Parental | 64 | IIYPGDSDTRYSPSFQG |
| HCDR2 | | |
| Parental | 65 | VGLDWNYNPLRY |
| HCDR3 | | |
| Parental | 66 | SGSSSDIGSNYVS |
| LCDR1 | | |
| Parental | 67 | DNNKRPS |
| LCDR2 | | |
| Parental | 68 | GTWDSRLGIAV |
| LCDR3 | | |
| IgG1 Heavy | 69 | ASTKGPSVFPLAPSSKSTSGGTAALGCLVKDYFPEPVTVSW |
| Chain constant | | NSGALTSGVHTFPAVLQSSGLYSLSSVVTVPSSSLGTQTYIC |
| region (YTE) | | NVNHKPSNTKVDKKVEPKSCDKTHTCPPCPAPELLGGPSVF |
| | | LFPPKPKDTLYITREPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKNQ |
| | | VSLTCLVKGFYPSDIAVEWESNGQPENNYKTTPPVLDSDGS |
| | | FFLYSKLTVDKSRWQQGNVFSCSVMHEALHNHYTQKSLSL |
| | | SPGK |
| Lambda Light | 70 | GQPKAAPSVTLFPPSSEELQANKATLVCLVSDFYPGAVTVA |
| Chain constant | | WKADGSPVKVGVETTKPSKQSNNKYAASSYLSLTPEQWKS |
| region | | HRSYSCRVTHEGSTVEKTVAPTECS |

1. An antigen binding protein or an antigen binding fragment thereof which specifically binds thyroid-stimulating hormone receptor (TSHR), wherein the antigen binding protein or an antigen binding fragment thereof comprises a heavy chain variable (VH) domain and a light chain variable (VL) domain, wherein:
a. the VH domain comprises a HCDR1 sequence comprising the amino acid sequence of SEQ ID NO: 21, a HCDR2 sequence comprising the amino acid sequence of SEQ ID NO: 22, and a HCDR3 sequence comprising the amino acid sequence of SEQ ID NO: 23; and the VL domain comprises a LCDR1 sequence comprising the amino acid sequence of SEQ ID NO: 24, a LCDR2 sequence comprising the amino acid sequence of SEQ ID NO: 25, and a LCDR3 sequence comprising the amino acid sequence of SEQ ID NO: 26;
b. the VH domain comprises a HCDR1 sequence comprising the amino acid sequence of SEQ ID NO: 1, a HCDR2 sequence comprising the amino acid sequence of SEQ ID NO: 2, and a HCDR3 sequence comprising the amino acid sequence of SEQ ID NO: 3; and the VL domain comprises a LCDR1 sequence comprising the amino acid sequence of SEQ ID NO: 4, a LCDR2 sequence comprising the amino acid sequence of SEQ ID NO: 5, and a LCDR3 sequence comprising the amino acid sequence of SEQ ID NO: 6;
c. the VH domain comprises a HCDR1 sequence comprising the amino acid sequence of SEQ ID NO: 11, a HCDR2 sequence comprising the amino acid sequence of SEQ ID NO: 12, and a HCDR3 sequence comprising the amino acid sequence of SEQ ID NO: 13; and the VL domain comprises a LCDR1 sequence comprising the amino acid sequence of SEQ ID NO: 14, a LCDR2 sequence comprising the amino acid sequence of SEQ ID NO: 15, and a LCDR3 sequence comprising the amino acid sequence of SEQ ID NO: 16;
d. the VH domain comprises a HCDR1 sequence comprising the amino acid sequence of SEQ ID NO: 31, a HCDR2 sequence comprising the amino acid sequence of SEQ ID NO: 32, and a HCDR3 sequence comprising the amino acid sequence of SEQ ID NO: 33; and the VL domain comprises a LCDR1 sequence comprising the amino acid sequence of SEQ ID NO: 34, a LCDR2 sequence comprising the amino acid sequence of SEQ ID NO: 35, and a LCDR3 sequence comprising the amino acid sequence of SEQ ID NO: 36;
e. the VH domain comprises a HCDR1 sequence comprising the amino acid sequence of SEQ ID NO: 41, a HCDR2 sequence comprising the amino acid sequence of SEQ ID NO: 42, and a HCDR3 sequence comprising the amino acid sequence of SEQ ID NO: 43; and the VL domain comprises a LCDR1 sequence comprising the amino acid sequence of SEQ ID NO: 44, a LCDR2 sequence comprising the amino acid sequence of SEQ ID NO: 45, and a LCDR3 sequence comprising the amino acid sequence of SEQ ID NO: 46; or
f. the VH domain comprises a HCDR1 sequence comprising the amino acid sequence of SEQ ID NO: 51, a HCDR2 sequence comprising the amino acid sequence of SEQ ID NO: 52, and a HCDR3 sequence comprising the amino acid sequence of SEQ ID NO: 53; and the VL domain comprises a LCDR1 sequence comprising the amino acid sequence of SEQ ID NO: 54, a LCDR2 sequence comprising the amino acid sequence of SEQ ID NO: 55, and a LCDR3 sequence comprising the amino acid sequence of SEQ ID NO: 56.

2. The antigen binding protein or antigen binding fragment thereof of claim 1, wherein:
a. the VH comprises an amino acid sequence at least 90% identical to SEQ ID NO: 27 and the VL comprises an amino acid sequence at least 90% identical to SEQ ID NO: 28;
b. the VH comprises an amino acid sequence at least 90% identical to SEQ ID NO: 7 and the VL comprises an amino acid sequence at least 90% identical to SEQ ID NO: 8;
c. the VH comprises an amino acid sequence at least 90% identical to SEQ ID NO: 17 and the VL comprises an amino acid sequence at least 90% identical to SEQ ID NO: 18;
d. the VH comprises an amino acid sequence at least 90% identical to SEQ ID NO: 37 and the VL comprises an amino acid sequence at least 90% identical to SEQ ID NO: 38;
e. the VH comprises an amino acid sequence at least 90% identical to SEQ ID NO: 47 and the VL comprises an amino acid sequence at least 90% identical to SEQ ID NO: 48; or
f. the VH comprises an amino acid sequence at least 90% identical to SEQ ID NO: 57 and the VL comprises an amino acid sequence at least 90% identical to SEQ ID NO: 58.

3. The antigen binding protein or antigen binding fragment thereof of claim 1, wherein:
a. the VH comprises an amino acid sequence of SEQ ID NO: 27 and the VL comprises an amino acid sequence of SEQ ID NO: 28;
b. the VH comprises an amino acid sequence of SEQ ID NO: 7 and the VL comprises an amino acid sequence of SEQ ID NO: 8;
c. the VH comprises an amino acid sequence of SEQ ID NO: 17 and the VL comprises an amino acid sequence of SEQ ID NO: 18;
d. the VH comprises an amino acid sequence of SEQ ID NO: 37 and the VL comprises an amino acid sequence of SEQ ID NO: 38;
e. the VH comprises an amino acid sequence of SEQ ID NO: 47 and the VL comprises an amino acid sequence of SEQ ID NO: 48; or
f. the VH comprises an amino acid sequence of SEQ ID NO: 57 and the VL comprises an amino acid sequence of SEQ ID NO: 58.

4. The antigen binding protein or the antigen binding fragment thereof of claim 1, wherein the antigen binding protein or the antigen binding fragment thereof further comprises an immunoglobulin Fc domain or variant thereof.

5. The antigen binding protein or the antigen binding fragment thereof of claim 4, wherein the Fc domain or variant thereof comprises a first Fc heavy chain and a second Fc heavy chain.

6. The antigen binding protein or the antigen binding fragment thereof of claim 5, wherein one or both of the first Fc heavy chain and second Fc heavy chain comprises one or more mutations to increase serum half-life.

7. The antigen binding protein or the antigen binding fragment thereof of claim 6, wherein one or both of the first Fc heavy chain and second Fc heavy chain comprises one or more substitutions at amino acid positions 252, 254, and/or 256, according to EU numbering.

8. The antigen binding protein or the antigen binding fragment thereof of claim 7,
wherein the substitution at amino acid position 252 is a tyrosine (Y),
wherein the substitution at amino acid position 254 is a threonine (T), and
wherein the substitution at amino acid position 256 is a glutamic acid (E).

9. The antigen binding protein or the antigen binding fragment thereof of claim 6, wherein the Fc heavy chain comprises an amino acid sequence of SEQ ID NO: 69.

10. The antigen binding protein or the antigen binding fragment thereof of claim 1, wherein the antigen binding protein or the antigen binding fragment thereof comprises:
a. a heavy chain polypeptide comprising an amino acid sequence at least 90% identical to SEQ ID NO: 29, and a light chain polypeptide comprising an amino acid sequence at least 90% identical to SEQ ID NO: 30;
b. a heavy chain polypeptide comprising an amino acid sequence at least 90% identical to SEQ ID NO: 9, and a light chain polypeptide comprising an amino acid sequence at least 90% identical to SEQ ID NO: 10;
c. a heavy chain polypeptide comprising an amino acid sequence at least 90% identical to SEQ ID NO: 19, and a light chain polypeptide comprising an amino acid sequence at least 90% identical to SEQ ID NO: 20;
d. a heavy chain polypeptide comprising an amino acid sequence at least 90% identical to SEQ ID NO: 39, and a light chain polypeptide comprising an amino acid sequence at least 90% identical to SEQ ID NO: 40;
e. a heavy chain polypeptide comprising an amino acid sequence at least 90% identical to SEQ ID NO: 49, and a light chain polypeptide comprising an amino acid sequence at least 90% identical to SEQ ID NO: 50; or
f. a heavy chain polypeptide comprising an amino acid sequence at least 90% identical to SEQ ID NO: 69, and a light chain polypeptide comprising an amino acid sequence at least 90% identical to SEQ ID NO: 70.

11. The antigen binding protein or the antigen binding fragment thereof of claim 1, wherein the antigen binding protein or the antigen binding fragment thereof comprises:
a. a heavy chain polypeptide comprising SEQ ID NO: 29, and a light chain polypeptide comprising SEQ ID NO: 30;
b. a heavy chain polypeptide comprising SEQ ID NO: 9, and a light chain polypeptide comprising SEQ ID NO: 10;
c. a heavy chain polypeptide comprising SEQ ID NO: 19, and a light chain polypeptide comprising SEQ ID NO: 20;
d. a heavy chain polypeptide comprising SEQ ID NO: 39, and a light chain polypeptide comprising SEQ ID NO: 40;
e. a heavy chain polypeptide comprising SEQ ID NO: 49, and a light chain polypeptide comprising SEQ ID NO: 50; or
f. a heavy chain polypeptide comprising SEQ ID NO: 69, and a light chain polypeptide comprising SEQ ID NO: 70.

12. The antigen binding protein or the antigen binding fragment thereof of claim 6, wherein one or both of the first Fc heavy chain and second Fc heavy chain comprises one or more substitutions at amino acid positions 428 and/or 434, according to EU numbering, optionally wherein the substitution at amino acid position 428 is a leucine (L), and wherein the substitution at amino acid position 434 is a serine (S).

13. (canceled)

14. The antigen binding protein or the antigen binding fragment thereof of claim 1, wherein the antigen binding protein or antigen binding fragment thereof has reduced binding, optionally at least about 2-fold reduced binding, to CD80 compared to an antigen binding protein comprising a VH domain of SEQ ID NO: 61 and a VL domain of SEQ ID NO: 62, optionally wherein binding to CD80 is determined by detecting binding of the antigen binding protein or the antigen binding fragment thereof to a cell that overexpresses CD80.

15-16. (canceled)

17. The antigen binding protein or the antigen binding fragment thereof of claim 1, wherein the antigen binding protein or the antigen binding fragment thereof has a higher melting temperature compared to an antigen binding protein comprising a VH domain of SEQ ID NO: 61 and a VL domain of SEQ ID NO: 62.

18. The antigen binding protein or the antigen binding fragment thereof of claim 1, wherein the antigen binding protein or the antigen binding fragment thereof has a melting temperature that is at least about 2° C. higher compared to an antigen binding protein comprising a VH domain of SEQ ID NO: 61 and a VL domain of SEQ ID NO: 62, optionally wherein the melting temperature is determined by differential scanning fluorimetry (DSF).

19. (canceled)

20. The antigen binding protein or the antigen binding fragment thereof of claim 1, wherein the antigen binding protein or the antigen binding fragment thereof blocks TSHR autoantibodies from binding to TSHR.

21. The antigen binding protein or the antigen binding fragment thereof of claim 1, wherein the antigen binding protein or the antigen binding fragment thereof decreases an autoimmune antibody response compared to an antigen binding protein comprising a VH domain of SEQ ID NO: 61 and a VL domain of SEQ ID NO: 62.

22. The antigen binding protein or the antigen binding fragment thereof of claim 1, wherein the antigen binding protein or the antigen binding fragment thereof has a reduced anti-drug antibody response compared to an antigen binding protein comprising a VH domain of SEQ ID NO: 61 and a VL domain of SEQ ID NO: 62.

23. The antigen binding protein or the antigen binding fragment thereof of claim 1, wherein the antigen binding protein or the antigen binding fragment thereof has reduced immunogenicity compared to an antigen binding protein comprising a VH domain of SEQ ID NO: 61 and a VL domain of SEQ ID NO: 62, optionally wherein the immunogenicity is determined by measuring the number of HLA class II restricted epitopes in the antigen binding protein or the antigen binding fragment thereof.

24. (canceled)

25. The antigen binding protein or the antigen binding fragment thereof of claim 23, wherein the HLA class II restricted epitopes are any one or more of HLA-DRB1, HLA-DRB3, HLA-DRB4, HLA-DRB5, HLA-DQ, and HLA-DP.

26. A pharmaceutical composition comprising the antigen binding protein or the antigen binding fragment thereof of claim 1 and a pharmaceutically acceptable carrier.

27. An isolated nucleic acid molecule encoding the antigen binding protein or the antigen binding fragment thereof of claim 1.

28. An expression vector comprising the nucleic acid molecule of claim 27.

29. A host cell comprising the expression vector of claim 28.

30. A method of treating or preventing a thyroid stimulating hormone receptor (TSHR)-related disease in a subject, comprising administering to a subject in need thereof an antigen binding protein or an antigen binding fragment thereof which specifically binds thyroid-stimulating hormone receptor (TSHR), wherein the antigen binding protein or an antigen binding fragment thereof comprises a heavy chain variable (VH) domain and a light chain variable (VL) domain, wherein:
a. the VH domain comprises a HCDR1 sequence comprising the amino acid sequence of SEQ ID NO: 21, a HCDR2 sequence comprising the amino acid sequence of SEQ ID NO: 22, and a HCDR3 sequence comprising the amino acid sequence of SEQ ID NO: 23; and the VL domain comprises a LCDR1 sequence comprising the amino acid sequence of SEQ ID NO: 24, a LCDR2 sequence comprising the amino acid sequence of SEQ ID NO: 25, and a LCDR3 sequence comprising the amino acid sequence of SEQ ID NO: 26;
b. the VH domain comprises a HCDR1 sequence comprising the amino acid sequence of SEQ ID NO: 1, a HCDR2 sequence comprising the amino acid sequence of SEQ ID NO: 2, and a HCDR3 sequence comprising the amino acid sequence of SEQ ID NO: 3; and the VL domain comprises a LCDR1 sequence comprising the amino acid sequence of SEQ ID NO: 4, a LCDR2 sequence comprising the amino acid sequence of SEQ ID NO: 5, and a LCDR3 sequence comprising the amino acid sequence of SEQ ID NO: 6;
c. the VH domain comprises a HCDR1 sequence comprising the amino acid sequence of SEQ ID NO: 11, a HCDR2 sequence comprising the amino acid sequence of SEQ ID NO: 12, and a HCDR3 sequence comprising the amino acid sequence of SEQ ID NO: 13; and the VL domain comprises a LCDR1 sequence comprising the amino acid sequence of SEQ ID NO: 14, a LCDR2 sequence comprising the amino acid sequence of SEQ ID NO: 15, and a LCDR3 sequence comprising the amino acid sequence of SEQ ID NO: 16;
d. the VH domain comprises a HCDR1 sequence comprising the amino acid sequence of SEQ ID NO: 31, a HCDR2 sequence comprising the amino acid sequence of SEQ ID NO: 32, and a HCDR3 sequence comprising the amino acid sequence of SEQ ID NO: 33; and the VL domain comprises a LCDR1 sequence comprising the amino acid sequence of SEQ ID NO: 34, a LCDR2 sequence comprising the amino acid sequence of SEQ ID NO: 35, and a LCDR3 sequence comprising the amino acid sequence of SEQ ID NO: 36;
e. the VH domain comprises a HCDR1 sequence comprising the amino acid sequence of SEQ ID NO: 41, a HCDR2 sequence comprising the amino acid sequence of SEQ ID NO: 42, and a HCDR3 sequence comprising the amino acid sequence of SEQ ID NO: 43; and the VL domain comprises a LCDR1 sequence comprising the amino acid sequence of SEQ ID NO: 44, a LCDR2 sequence comprising the amino acid sequence of SEQ ID NO: 45, and a LCDR3 sequence comprising the amino acid sequence of SEQ ID NO: 46; or
f. the VH domain comprises a HCDR1 sequence comprising the amino acid sequence of SEQ ID NO: 51, a HCDR2 sequence comprising the amino acid sequence of SEQ ID NO: 52, and a HCDR3 sequence comprising the amino acid sequence of SEQ ID NO: 53; and the VL domain comprises a LCDR1 sequence comprising the amino acid sequence of SEQ ID NO: 54, a LCDR2 sequence comprising the amino acid sequence of SEQ ID NO: 55, and a LCDR3 sequence comprising the amino acid sequence of SEQ ID NO: 56, thereby treating or preventing the TSHR-related disease in the subject.

31. The method of claim 30, wherein the TSHR-related disease is an autoimmune disease, Graves' disease, thyrotoxicosis, and/or cancer.

32-34. (canceled)

35. A method of treating an autoimmune disease associated with autoantibodies to thyroid stimulating hormone receptor (TSHR) in a subject, comprising administering to a subject in need thereof the antigen binding protein or antigen binding fragment thereof of claim 1, thereby treating or preventing the autoimmune disease associated with autoantibodies to TSHR in the subject.

36. A chimeric antigen receptor (CAR) comprising a TSHR-binding domain comprising an antigen binding protein or an antigen binding fragment thereof which specifically binds thyroid-stimulating hormone receptor (TSHR), wherein the antigen binding protein or an antigen binding fragment thereof comprises a heavy chain variable (VH) domain and a light chain variable (VL) domain, wherein:
a. the VH domain comprises a HCDR1 sequence comprising the amino acid sequence of SEQ ID NO: 21, a HCDR2 sequence comprising the amino acid sequence of SEQ ID NO: 22, and a HCDR3 sequence comprising the amino acid sequence of SEQ ID NO: 23; and the VL domain comprises a LCDR1 sequence comprising the amino acid sequence of SEQ ID NO: 24, a LCDR2 sequence comprising the amino acid sequence of SEQ ID NO: 25, and a LCDR3 sequence comprising the amino acid sequence of SEQ ID NO: 26;
b. the VH domain comprises a HCDR1 sequence comprising the amino acid sequence of SEQ ID NO: 1, a HCDR2 sequence comprising the amino acid sequence of SEQ ID NO: 2, and a HCDR3 sequence comprising the amino acid sequence of SEQ ID NO: 3; and the VL domain comprises a LCDR1 sequence comprising the amino acid sequence of SEQ ID NO: 4, a LCDR2 sequence comprising the amino acid sequence of SEQ ID NO: 5, and a LCDR3 sequence comprising the amino acid sequence of SEQ ID NO: 6;
c. the VH domain comprises a HCDR1 sequence comprising the amino acid sequence of SEQ ID NO: 11, a HCDR2 sequence comprising the amino acid sequence of SEQ ID NO: 12, and a HCDR3 sequence comprising the amino acid sequence of SEQ ID NO: 13; and the VL domain comprises a LCDR1 sequence comprising the amino acid sequence of SEQ ID NO: 14, a LCDR2 sequence comprising the amino acid sequence of SEQ ID NO: 15, and a LCDR3 sequence comprising the amino acid sequence of SEQ ID NO: 16;
d. the VH domain comprises a HCDR1 sequence comprising the amino acid sequence of SEQ ID NO: 31, a HCDR2 sequence comprising the amino acid sequence of SEQ ID NO: 32, and a HCDR3 sequence comprising the amino acid sequence of SEQ ID NO: 33; and the VL domain comprises a LCDR1 sequence comprising the amino acid sequence of SEQ ID NO: 34, a LCDR2 sequence comprising the amino acid sequence of SEQ ID NO: 35, and a LCDR3 sequence comprising the amino acid sequence of SEQ ID NO: 36;
e. the VH domain comprises a HCDR1 sequence comprising the amino acid sequence of SEQ ID NO: 41, a HCDR2 sequence comprising the amino acid sequence of SEQ ID NO: 42, and a HCDR3 sequence comprising the amino acid sequence of SEQ ID NO: 43; and the VL domain comprises a LCDR1 sequence comprising the amino acid sequence of SEQ ID NO: 44, a LCDR2 sequence comprising the amino acid sequence of SEQ ID NO: 45, and a LCDR3 sequence comprising the amino acid sequence of SEQ ID NO: 46; or
f. the VH domain comprises a HCDR1 sequence comprising the amino acid sequence of SEQ ID NO: 51, a HCDR2 sequence comprising the amino acid sequence of SEQ ID NO: 52, and a HCDR3 sequence comprising the amino acid sequence of SEQ ID NO: 53; and the VL domain comprises a LCDR1 sequence comprising the amino acid sequence of SEQ ID NO: 54, a LCDR2 sequence comprising the amino acid sequence of SEQ ID NO: 55, and a LCDR3 sequence comprising the amino acid sequence of SEQ ID NO: 56.

37. A cell comprising the CAR of claim 36.

38-43. (canceled)

44. An antibody drug conjugate (ADC) comprising an antigen binding protein or an antigen binding fragment thereof which specifically binds thyroid-stimulating hormone receptor (TSHR), wherein the antigen binding protein or an antigen binding fragment thereof comprises a heavy chain variable (VH) domain and a light chain variable (VL) domain, wherein:
a. the VH domain comprises a HCDR1 sequence comprising the amino acid sequence of SEQ ID NO: 21, a HCDR2 sequence comprising the amino acid sequence of SEQ ID NO: 22, and a HCDR3 sequence comprising the amino acid sequence of SEQ ID NO: 23; and the VL domain comprises a LCDR1 sequence comprising the amino acid sequence of SEQ ID NO: 24, a LCDR2 sequence comprising the amino acid sequence of SEQ ID NO: 25, and a LCDR3 sequence comprising the amino acid sequence of SEQ ID NO: 26;
b. the VH domain comprises a HCDR1 sequence comprising the amino acid sequence of SEQ ID NO: 1, a HCDR2 sequence comprising the amino acid sequence of SEQ ID NO: 2, and a HCDR3 sequence comprising the amino acid sequence of SEQ ID NO: 3; and the VL domain comprises a LCDR1 sequence comprising the amino acid sequence of SEQ ID NO: 4, a LCDR2 sequence comprising the amino acid sequence of SEQ ID NO: 5, and a LCDR3 sequence comprising the amino acid sequence of SEQ ID NO: 6;
c. the VH domain comprises a HCDR1 sequence comprising the amino acid sequence of SEQ ID NO: 11, a HCDR2 sequence comprising the amino acid sequence of SEQ ID NO: 12, and a HCDR3 sequence comprising the amino acid sequence of SEQ ID NO: 13; and the VL domain comprises a LCDR1 sequence comprising the amino acid sequence of SEQ ID NO: 14, a LCDR2 sequence comprising the amino acid sequence of SEQ ID NO: 15, and a LCDR3 sequence comprising the amino acid sequence of SEQ ID NO: 16;
d. the VH domain comprises a HCDR1 sequence comprising the amino acid sequence of SEQ ID NO: 31, a HCDR2 sequence comprising the amino acid sequence of SEQ ID NO: 32, and a HCDR3 sequence comprising the amino acid sequence of SEQ ID NO: 33; and the VL domain comprises a LCDR1 sequence comprising the amino acid sequence of SEQ ID NO: 34, a LCDR2 sequence comprising the amino acid sequence of SEQ ID NO: 35, and a LCDR3 sequence comprising the amino acid sequence of SEQ ID NO: 36;
e. the VH domain comprises a HCDR1 sequence comprising the amino acid sequence of SEQ ID NO: 41, a HCDR2 sequence comprising the amino acid sequence of SEQ ID NO: 42, and a HCDR3 sequence comprising the amino acid sequence of SEQ ID NO: 43; and the VL domain comprises a LCDR1 sequence comprising the amino acid sequence of SEQ ID NO: 44, a LCDR2 sequence comprising the amino acid sequence of SEQ ID NO: 45, and a LCDR3 sequence comprising the amino acid sequence of SEQ ID NO: 46; or
f. the VH domain comprises a HCDR1 sequence comprising the amino acid sequence of SEQ ID NO: 51, a HCDR2 sequence comprising the amino acid sequence of SEQ ID NO: 52, and a HCDR3 sequence comprising the amino acid sequence of SEQ ID NO: 53; and the VL domain comprises a LCDR1 sequence comprising the amino acid sequence of SEQ ID NO: 54, a LCDR2 sequence comprising the amino acid sequence of SEQ ID NO: 55, and a LCDR3 sequence comprising the amino acid sequence of SEQ ID NO: 56, conjugated to a drug, optionally wherein the drug is a radioisotope or to a therapeutic small molecule.

45-51. (canceled)


SEQUENCE LISTING (74 sequences)

SEQ ID NO: 1 - protein, 8 aa, synthetic construct
GYSLTDNW

SEQ ID NO: 2 - protein, 8 aa, synthetic construct
IYPGDSDT

SEQ ID NO: 3 - protein, 12 aa, synthetic construct
VGLDWNYNPLRY

SEQ ID NO: 4 - protein, 8 aa, synthetic construct
LSDIGSNY

SEQ ID NO: 5 - protein, 7 aa, synthetic construct
DNNKRPS

SEQ ID NO: 6 - protein, 11 aa, synthetic construct
GTWDSRLGIAV

SEQ ID NO: 7 - protein, 119 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 8 - protein, 110 aa, synthetic construct
QSVLTQPPSVSAAPGQKVTISCSGSLSDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 9 - protein, 449 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAIYYCVGLDWNYNPLRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, synthetic construct
QSVLTQPPSVSAAPGQKVTISCSGSLSDIGSNYVSWYQQLPGTAPKLLIYDNNKRPSGIPDRFSGSKSGTSATLGITGLQTGDEADYYCGTWDSRLGIAVFGGGTQLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 11 - protein, 8 aa, synthetic construct
GYSLTDNW

SEQ ID NO: 12 - protein, 8 aa, synthetic construct
IYPGDSDT

SEQ ID NO: 13 - protein, 12 aa, synthetic construct
VGLDWNYNPLRY

SEQ ID NO: 14 - protein, 8 aa, synthetic construct
LRDIGSNY

SEQ ID NO: 15 - protein, 7 aa, synthetic construct
DNNKRPS

SEQ ID NO: 16 - protein, 11 aa, synthetic construct
GTWDSRLGIAV

SEQ ID NO: 17 - protein, 119 aa, synthetic construct; identical to SEQ ID NO: 7

SEQ ID NO: 18 - protein, 110 aa, synthetic construct
QSVLTQPPSVSAAPGQKVTISCSGSLRDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 19 - protein, 449 aa, synthetic construct; identical to SEQ ID NO: 9

SEQ ID NO: 20 - protein, 216 aa, synthetic construct
QSVLTQPPSVSAAPGQKVTISCSGSLRDIGSNYVSWYQQLPGTAPKLLIYDNNKRPSGIPDRFSGSKSGTSATLGITGLQTGDEADYYCGTWDSRLGIAVFGGGTQLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 21 - protein, 8 aa, synthetic construct
GYSLTDNW

SEQ ID NO: 22 - protein, 8 aa, synthetic construct
IYPGDSTT

SEQ ID NO: 23 - protein, 12 aa, synthetic construct
VGLDWNYNPLRY

SEQ ID NO: 24 - protein, 8 aa, synthetic construct
YSDIGSNY

SEQ ID NO: 25 - protein, 7 aa, synthetic construct
DNNKRPS

SEQ ID NO: 26 - protein, 11 aa, synthetic construct
GTWDSRLGIAV

SEQ ID NO: 27 - protein, 119 aa, synthetic construct
EVQLVQSGAEVKKPGASVKVSCKASGYSLTDNWIGWVRQAPGQGLEWMGIIYPGDSTTRYAQKFQGRVTMTADKSTSTVYMELSSLRSEDTAVYYCVGLDWNYNPLRYWGQGTLVTVSS

SEQ ID NO: 28 - protein, 110 aa, synthetic construct
QSVLTQPPSVSGAPGQRVTISCSGSYSDIGSNYVSWYQQLPGTAPKLLIYDNNKRPSGVPDRFSGSKSGTSASLAITGLQAEDEADYYCGTWDSRLGIAVFGGGTKLTVL

SEQ ID NO: 29 - protein, 449 aa, synthetic construct
EVQLVQSGAEVKKPGASVKVSCKASGYSLTDNWIGWVRQAPGQGLEWMGIIYPGDSTTRYAQKFQGRVTMTADKSTSTVYMELSSLRSEDTAVYYCVGLDWNYNPLRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 216 aa, synthetic construct
QSVLTQPPSVSGAPGQRVTISCSGSYSDIGSNYVSWYQQLPGTAPKLLIYDNNKRPSGVPDRFSGSKSGTSASLAITGLQAEDEADYYCGTWDSRLGIAVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 31 - protein, 8 aa, synthetic construct
GYSLTDNW

SEQ ID NO: 32 - protein, 8 aa, synthetic construct
IYPGDSDT

SEQ ID NO: 33 - protein, 12 aa, synthetic construct
VGLDWNYNPLRY

SEQ ID NO: 34 - protein, 8 aa, synthetic construct
LRDIGSNY

SEQ ID NO: 35 - protein, 7 aa, synthetic construct
DNNKRPS

SEQ ID NO: 36 - protein, 11 aa, synthetic construct
GTWDSRLGIAV

SEQ ID NO: 37 - protein, 119 aa, synthetic construct
EVQLVQSGAEVKKPGASVKVSCKASGYSLTDNWIGWVRQAPGQGLEWMGIIYPGDSDTRYAQKFQGRVTMTADKSTSTVYMELSSLRSEDTAVYYCVGLDWNYNPLRYWGQGTLVTVSS

SEQ ID NO: 38 - protein, 110 aa, synthetic construct
QSVLTQPPSVSGAPGQRVTISCSGSLRDIGSNYVSWYQQLPGTAPKLLIYDNNKRPSGVPDRFSGSKSGTSASLAITGLQAEDEADYYCGTWDSRLGIAVFGGGTKLTVL

SEQ ID NO: 39 - protein, 449 aa, synthetic construct
EVQLVQSGAEVKKPGASVKVSCKASGYSLTDNWIGWVRQAPGQGLEWMGIIYPGDSDTRYAQKFQGRVTMTADKSTSTVYMELSSLRSEDTAVYYCVGLDWNYNPLRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 216 aa, synthetic construct
QSVLTQPPSVSGAPGQRVTISCSGSLRDIGSNYVSWYQQLPGTAPKLLIYDNNKRPSGVPDRFSGSKSGTSASLAITGLQAEDEADYYCGTWDSRLGIAVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 41 - protein, 8 aa, synthetic construct
GYSLTDNW

SEQ ID NO: 42 - protein, 8 aa, synthetic construct
IYPGDSDT

SEQ ID NO: 43 - protein, 12 aa, synthetic construct
VGLDWNYNPLRY

SEQ ID NO: 44 - protein, 8 aa, synthetic construct
SSNVGSNY

SEQ ID NO: 45 - protein, 7 aa, synthetic construct
DNNYVPS

SEQ ID NO: 46 - protein, 11 aa, synthetic construct
GTWDSRLDAAV

SEQ ID NO: 47 - protein, 119 aa, synthetic construct; identical to SEQ ID NO: 37

SEQ ID NO: 48 - protein, 110 aa, synthetic construct
QSVLTQPPSVSGAPGQRVTISCSGSSSNVGSNYVSWYQQLPGTAPKLLIYDNNYVPSGVPDRFSGSKSGTSASLAITGLQAEDEADYYCGTWDSRLDAAVFGGGTKLTVL

SEQ ID NO: 49 - protein, 449 aa, synthetic construct; identical to SEQ ID NO: 39

SEQ ID NO: 50 - protein, 216 aa, synthetic construct
QSVLTQPPSVSGAPGQRVTISCSGSSSNVGSNYVSWYQQLPGTAPKLLIYDNNYVPSGVPDRFSGSKSGTSASLAITGLQAEDEADYYCGTWDSRLDAAVFGGGTKLTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 51 - protein, 8 aa, synthetic construct
GYHLTHHW

SEQ ID NO: 52 - protein, 8 aa, synthetic construct
IYPGDSDT

SEQ ID NO: 53 - protein, 12 aa, synthetic construct
VGLDWNYNPLRY

SEQ ID NO: 54 - protein, 8 aa, synthetic construct
LSDIGSNY

SEQ ID NO: 55 - protein, 7 aa, synthetic construct
DNNKRPS

SEQ ID NO: 56 - protein, 11 aa, synthetic construct
GTWDSRLGIAV

SEQ ID NO: 57 - protein, 119 aa, synthetic construct
EVQLVQSGAEVKKPGESLKISCKASGYHLTHHWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 58 - protein, 110 aa, synthetic construct; identical to SEQ ID NO: 8

SEQ ID NO: 59 - protein, 449 aa, synthetic construct
EVQLVQSGAEVKKPGESLKISCKASGYHLTHHWIGWVRQKPGKGLEWMGIIYPGDSDTRYSPSFQGQVTISADKSISTAYLQWSSLKASDTAIYYCVGLDWNYNPLRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 216 aa, synthetic construct; identical to SEQ ID NO: 10

SEQ ID NO: 61 - protein, 119 aa, synthetic construct [sequence omitted]

SEQ ID NO: 62 - protein, 110 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVL

SEQ ID NO: 63 - protein, 10 aa, synthetic construct
GYSLTDNWIG

SEQ ID NO: 64 - protein, 17 aa, synthetic construct
IIYPGDSDTRYSPSFQG

SEQ ID NO: 65 - protein, 12 aa, synthetic construct
VGLDWNYNPLRY

SEQ ID NO: 66 - protein, 13 aa, synthetic construct
SGSSSDIGSNYVS

SEQ ID NO: 67 - protein, 7 aa, synthetic construct
DNNKRPS

SEQ ID NO: 68 - protein, 11 aa, synthetic construct
GTWDSRLGIAV

SEQ ID NO: 69 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 106 aa, synthetic construct
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 71 - protein, 119 aa, synthetic construct; identical to SEQ ID NO: 7

SEQ ID NO: 72 - protein, 110 aa, synthetic construct
QSVLTQPPSVSAAPGQKVTISCSGSSSDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 73 - protein, 119 aa, synthetic construct; identical to SEQ ID NO: 37

SEQ ID NO: 74 - protein, 110 aa, synthetic construct
QSVLTQPPSVSGAPGQRVTISCSGSSSDIGSNYVSWYQQLPGTAPKLLIYDNNKRPSGVPDRFSGSKSGTSASLAITGLQAEDEADYYCGTWDSRLGIAVFGGGTKLTVL